(19) United States
(12) Patent Application Publication
(10) Pub. No.: US 2024/0071062 A1
(43) Pub. Date: Feb. 29, 2024

(54) MODEL FUSION SYSTEM

(71) Applicant: JIVA.AI LIMITED, Cardiff (GB)

(72) Inventor: Manish Amritbhai PATEL, Northwood (GB)

(21) Appl. No.: 18/490,903

(22) Filed: Oct. 20, 2023

Related U.S. Application Data
(63) Continuation of application No. PCT/GB2022/050764, filed on Mar. 28, 2022.

(30) Foreign Application Priority Data
Apr. 23, 2021 (GB) 2105835.9

(51) Int. Cl. G06V 10/80; G06T 7/00; G06V 10/774; G06V 10/776; G06V 10/82; G16H 30/40; G16H 50/20

(52) U.S. Cl. CPC G06V 10/80; G06T 7/0012; G06V 10/774; G06V 10/776; G06V 10/82; G16H 30/40; G16H 50/20; G06T 2207/10081; G06T 2207/10088; G06T 2207/10116; G06T 2207/10132; G06T 2207/20081; G06T 2207/20084; G06T 2207/30008; G06T 2207/30056; G06T 2207/30081; G06T 2207/30096; G06V 2201/03

(57) ABSTRACT

Fusing or integrating at least two agent model(s) for modelling a complex system, each agent model comprising: a plurality of agent system (AS) node(s), where each of the AS node(s) comprise a plurality of agent units (AUs), and a set of AS rules governing the plurality of AUs, each AU of the plurality of AUs is connected to at least one other AU of the plurality of AUs, an input layer comprising a set of AS nodes of the plurality of AS node(s) an output layer comprising at least one AS node of the plurality of AS node(s) and one or more intermediate layer(s). Each of the intermediate layer(s) comprising another set of AS node(s) of the plurality of AS node(s). Each agent model is trained to model one or more portion(s) of the complex system using a corresponding labelled training dataset.

### CROSS-REFERENCE TO RELATED APPLICATIONS

This is a bypass continuation of International PCT Application No. PCT/GB2022/050764, filed on Mar. 28, 2022, which claims priority to United Kingdom Patent Application No. 2105835.9, filed on Apr. 23, 2021 which are incorporated by reference herein in their entirety.

### FIELD

The present application relates to an apparatus, system and method of generating a fusion or integrated model from multiple models for modelling a complex technical system, each model configured for modelling one or more aspects or portions of the complex technical system.

### BACKGROUND

Machine learning (ML) techniques have been employed for some time now, especially in the medical industry to detect tricky or hard to spot afflictions. For example predicting and/or detecting disease and/or state of a subject based on one or more data sources including data associated with the subject such as, without limitation, for example one or more medical or personal data sources with data associated with the subject including, without limitation for example, medical imaging of the subject; medical records of the subject; lifestyle and/or environmental data associated with the subject; medical test results/biopsies/bloodwork of the subject and/or any other type of medical data collected in relation to the subject and the like. For example, predicting and/or detecting where a subject has tumours and/or, if positive, where the tumour locations on medical imaging such as, without limitation, for example magnetic resonance imaging (MRI) scan image(s) or X-ray scan image(s). In another example, predicting and/or detecting hairline-fractures of the bone from X-ray scan image(s) of the subject. Recent advances in the area of image recognition, in particular towards the development of convolutional neural networks (CNN) with deep learning techniques and the like, has helped improve image resolution which in-turn has improved the model prediction and/or detection accuracy thereby adding immense value to the medical industry.

However, traditionally the process of supervised ML includes the steps of: preparing a labelled training data set, where the labelled training data set is composed of many feature sets and annotated with labels, where each feature set can be transformed to a vector with each feature set being annotated with one or more labels. Each of the one or more labels includes data representative of the outcome that needs to be learned; transforming the labelled training data set into mathematical vector(s) that completely describe relevant data point (i.e. feature space). For example, filtering the data first for the points of interest, normalisation of the filtered data (e.g. fitting all numbers between 0 and 1, numeral, alphabets, statistical normalisation across the entire data set); building the ML model topology/algorithm. For example, in artificial neural networks (ANNs) one would build layers of perceptrons and modify permutations of parameters to zero in a solution that performs well and/or a ML models “output node” (the final rung of the topology) usually produces a vector that describes the label (i.e. the predictor); and feeding the data vector (e.g. the transformed data set) into the ML model algorithm and learning/optimising (e.g. by modifying the input vector) based on the output vector produced by the model.

However, one of the downsides to, for example, improving image quality or discovering new (raw) data insights, is that machine learning algorithms typically need to be retrained with the new data set (for example, with an improved image with better resolution). This is an extremely expensive and time-consuming process with no guarantee of success (in terms of accuracy of detection) because fewer data points are now available for training the ML model. This is particularly not helpful for deep learning networks because they typically need thousands/millions of samples to train accurately. As a result, it becomes difficult to choose a network model/structure that will ensure that the ML model does not introduce artefacts that are not present in the image/miss the pathological detail unseen during training and other drawbacks.

Researchers have tried to train multiple different ML models or use an ensemble of ML models that produces multiple outputs associated with an outcome, where a final output outcome is performed by simply performing a weighted combination of the multiple outputs in an attempt to improve accuracy of prediction, detection, classification of the complex system modelled by the multiple different ML models/ensemble. Although this simplified optimisation approach may initially seem to assist in improving accuracy of prediction, classification, and/or detection and the like, such approaches will still result in sub-optimal predictions, classifications, detections and the like.

There is a desire for a technology that provides a more jointly optimised approach that has the ability to take into account the structures and/or any relationships that may be apparent between different ML models that model one or more aspects of a complex system that further improves the predictions, classifications, and/or detections and the like in relation to said modelled complex system.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter. Variants and alternative features which facilitate the working of the invention and/or serve to achieve a substantially similar technical effect should be considered as falling into the scope of the invention disclosed herein.

The present disclosure provides apparatus, methods, process(es), systems, mechanisms, and/or methodologies/algorithmic solutions that enabling creation of integrated ML models for modelling a complex system where the integrated ML model is based on two or more original models configured for modelling an aspect of the complex system in which each original model for modelling an aspect of the complex system maintains its original predictive behaviours whilst the predictive ability of the whole is enhanced by virtue of its constituents. The apparatus, methods, process(es), systems, mechanisms, methodologies are based on optimising a rule-based multi-agent system (MAS) (which itself is an ML model), wherein agents operate on decimal or symbolic vectors, to create a generalised and abstract computation describing pattern recognition in the given data domain or domain area in which the complex system/problem resides including domains such as, without limitation, for example healthcare and/or medical fields, automotive and transportation, spacetech, fintech, manufacturing and/or extraction, mining/agriculture, research and the like or any other field/domain in which ML or AI may be applied for modelling/solving complex technical systems/problems and the like. Furthermore, there are provided apparatus, methods, process(es), systems, and/or mechanisms in which MAS models can be fused with one another in such a way that the individual MAS' computational ability is not compromised by the merge yet, as a whole, the MAS ensemble or fusion model is able to predict on the corresponding data domains.

In a first aspect, the present disclosure provides a computer implemented method of detecting a disease or state of a subject from one or more images of the subject, the method comprising: obtaining a fusion agent model configured for modelling the detection of the disease or state of the subject from one or more images of the subject, the fusion agent model derived from at least two agent model(s), each agent model trained to model the detection of the disease or state of a subject from a different imaging source, each agent model comprising: a plurality of agent system (AS) node(s), wherein each of the AS node(s) comprise: a plurality of agent units (AUs) and a set of AS rules governing the plurality of AUs, each AU of the plurality of AUs connected to at least one other AU of the plurality of AUs; an input layer comprising a set of AS nodes of the plurality of AS node(s); an output layer comprising at least one AS node of the plurality of AS node(s); and one or more intermediate layer(s), each of the intermediate layer(s) comprising another set of AS node(s) of the plurality of AS node(s); each agent model is trained to model the detection of the disease or state of a subject based on a corresponding labelled training dataset comprising images from, and said each agent model being adapted, during training, to form an agent rule base comprising one or more sets of agent system rules; and an agent network state comprising data representative of the interconnections between the AS nodes of the input, output and intermediate layer(s), wherein the agent rule base and agent network state are generated during training and configured for modelling said portion(s) of the complex system; wherein obtaining the fusion agent model further comprising: determining an intersecting rule set between the agent rule bases of the at least two agent models; merging said at least two agent models to form a fused agent model based on combining those one or more layer(s), AS node(s), and/or AU(s) of the at least two agent models that correspond to the intersecting rule set; and updating the fused agent model based on one or more validation and training labelled datasets associated with each of the at least two agent model(s) until the fused agent model is validly trained, wherein the trained fused agent model is the fusion agent model; inputting said one or more images of the subject to the fusion agent model for detecting the disease or state of the subject based on the input one or more images of the subject; and outputting data representative of an indication of whether the disease or state is detected from the one or more images of the subject.

As an option, the computer implemented method of the first aspect, wherein the complex system to be modelled is detection of prostate cancer of a subject that is modelled by a plurality of prostate cancer detection agent models, wherein each prostate cancer detection agent model is trained using a labelled training dataset comprising a plurality of labelled training data images of subjects in relation to detecting or recognising prostate cancer tumours from said labelled training data images, wherein each prostate cancer detection agent model uses a labelled training dataset based on images output from the same type of imaging system that is different to the imaging systems used in each of the other prostate cancer detection agent model of the plurality of prostate cancer agent models.

As another option, the computer implemented method of the first aspect, wherein each imaging system is a particular magnetic resonance imaging (MRI) system by a particular manufacturer.

Optionally, the computer implemented method of the first aspect, wherein the complex system is bone fracture detection of a subject that is modelled by a plurality of bone fracture detection agent models, wherein each bone fracture detection agent model is trained using a labelled training dataset comprising a plurality of labelled training data images of subjects in relation to detecting or recognising bone fractures from said labelled training data images, wherein each bone fracture detection agent model uses a labelled training dataset based on images output from the same type of imaging system, wherein each labelled training data item is annotated or labelled as to whether or not said subject of the plurality of subjects has a bone fracture, wherein each bone fracture detection agent model is trained in relation to images associated with different imaging systems.

As an option, the computer implemented method of the first aspect, wherein images are acquired via imaging systems or techniques based on at least one from the group of: magnetic resonance imaging, MRI; computer tomography, CT; ultrasound; or X-ray; or images from any other medical imaging system for use in detecting disease and/or state of a subject.

In a second aspect, the present disclosure provides a computer implemented method of detecting a disease or state of a subject from a plurality of data sources associated with the subject, the method comprising: obtaining a fusion agent model configured for modelling the detection of the disease or state of the subject from said plurality of data sources associated with the subject, the fusion agent model derived from at least two agent model(s), each agent model trained to model the detection of the disease or state of a subject from a different data source associated with the subject, each agent model comprising: a plurality of agent system node(s), wherein each of the AS node(s) comprise: a plurality of agent units, AUs, and a set of AS rules governing the plurality of AUs, each AU of the plurality of AUs connected to at least one other AU of the plurality of AUs; an input layer comprising a set of AS nodes of the plurality of AS node(s); an output layer comprising at least one AS node of the plurality of AS node(s); and one or more intermediate layer(s), each of the intermediate layer(s) comprising another set of AS node(s) of the plurality of AS node(s); each agent model is trained to model the detection of the disease or state of a subject based on a corresponding labelled training dataset derived from the corresponding data source associated with the subject, and said each agent model being adapted, during training, to form an agent rule base comprising one or more sets of agent system rules; and an agent network state comprising data representative of the interconnections between the AS nodes of the input, output and intermediate layer(s), wherein the agent rule base and agent network state are generated during training and configured for modelling said portion(s) or aspects of the complex system; wherein obtaining the fusion agent model further comprising: determining an intersecting rule set between the agent rule bases of the at least two agent models; merging said at least two agent models to form a fused agent model based on combining those one or more layer(s), AS node(s), and/or AU(s) of the at least two agent models that correspond to the intersecting rule set; and updating the fused agent model based on one or more validation and training labelled datasets associated with each of the at least two agent model(s) until the fused agent model is validly trained, wherein the trained fused agent model is the fusion agent model; inputting data representative of said one or more data sources associated with the subject to the fusion agent model for detecting the disease or state of the subject; and outputting data representative of an indication of whether the disease or state is detected from the input one or more data sources associated with the subject.

As an option, the computer implemented method of the second aspect, wherein the complex system to be modelled is liver disease detection of a subject that is modelled by a plurality of liver disease detection agent models, wherein each liver disease detection agent model is trained using a labelled training dataset derived from a different data source associated with a plurality of subjects, said each labelled training dataset comprising a plurality of labelled training data items based on the different data source and annotated in relation to whether or not said plurality of subjects have liver disease, said each trained liver disease detection agent model associated with a different, but related, aspect of the complex system of liver disease detection.

As another option, the computer implemented method of the second aspect, wherein the plurality of liver disease detection agent models comprises at least the liver disease detection agent models from the group of: a first liver disease detection agent model trained based on a labelled training dataset comprising a plurality of labelled training data items associated with a plurality of subjects, each training data item corresponding to data representative of lifestyle and/or ethnic background data of a subject of the plurality of subjects and annotated or labelled as to whether or not said subject of the plurality of subjects has liver disease; a second liver disease detection agent model trained based on a labelled training dataset comprising a plurality of labelled training data items associated with a plurality of subjects, each training data item corresponding to data representative of the genetics of a subject of the plurality of subjects and annotated or labelled as to whether or not said subject of the plurality of subjects has liver disease; a third liver disease detection agent model trained based on a labelled training dataset comprising a plurality of labelled training data items associated with a plurality of subjects, each training data item corresponding to data representative of one or more proteomic blood markers of a subject of the plurality of subjects and annotated or labelled as to whether or not said subject of the plurality of subjects has liver disease; a fourth liver disease detection agent model trained based on a labelled training dataset comprising a plurality of labelled training data items associated with a plurality of subjects, each training data item corresponding to data representative of medical history of a subject of the plurality of subjects and annotated or labelled as to whether or not said subject of the plurality of subjects has liver disease; a fifth liver disease detection agent model trained based on a labelled training dataset comprising a plurality of labelled training data items associated with a plurality of subjects, each training data item corresponding to data representative of a sonograph and/or imaging of the liver of a subject of the plurality of subjects and annotated or labelled as to whether or not said subject of the plurality of subjects has liver disease; and one or more other liver disease detection agent model(s), each trained based on a labelled training dataset comprising a plurality of labelled training data items associated with a plurality of subjects, each training data item corresponding to data representative of modelling another aspect of the complex system for diagnosing liver disease and annotated or labelled as to whether or not said subject of the plurality of subjects has liver disease.

In a third aspect, the present disclosure provides a computer implemented method of fusing or integrating at least two agent model(s) for modelling a complex system, each agent model comprising: a plurality of agent system node(s), wherein each of the AS node(s) comprise a plurality of agent units, AUs, and a set of AS rules governing the plurality of AUs, each AU of the plurality of AUs connected to at least one other AU of the plurality of AUs; an input layer comprising a set of AS nodes of the plurality of AS node(s) an output layer comprising at least one AS node of the plurality of AS node(s); and one or more intermediate layer(s), each of the intermediate layer(s) comprising another set of AS node(s) of the plurality of AS node(s); wherein each agent model is trained to model one or more portion(s) of the complex system using a corresponding labelled training dataset, said each agent model being adapted, during training, to form: an agent rule base comprising one or more sets of AS rules; and an agent network state comprising data representative of the interconnections between the AS nodes of the input, output and intermediate layer(s), wherein the agent rule base and agent network state are generated during training and configured for modelling said portion(s) of the complex system; the method comprising: determining an intersecting rule set between the agent rule bases of at least a first trained agent model and a second trained agent model; merging said at least first and second trained agent models to form an integrated agent model based on combining those one or more layer(s), AS node(s), and/or AU(s) of the first and second trained agent models that correspond to the intersecting rule set; and updating the integrated agent model based on one or more validation and training labelled datasets associated with each of the at least first and second trained agent model(s) until the integrated model is validly trained.

As an option, the computer implemented method of any of the first, second and/or third aspects, wherein the complex system is modelled by a plurality of agent model(s), each agent model of the plurality of agent model(s) configured to model a different portion of the complex system, the method further comprising: determining an intersecting rule set between two or more of the agent rule bases of the plurality of agent model(s), wherein the agent rule base of each of the plurality of agent model(s) intersects with at least one other agent rule base of another of the plurality of agent model(s); and merging said plurality of agent models to form an integrated agent model based on combining those one or more layer(s), AS node(s), and/or AU(s) of each of the plurality of agent models that correspond to the intersecting rule set; and updating the integrated agent model based on one or more validation and training labelled datasets associated with each of the at least first and second trained agent model(s) until the integrated model is validly trained.

As another option, the computer implemented method of any of the first, second and/or third aspects, wherein the complex system is modelled by a plurality of agent model(s), each agent model of the plurality of agent model(s) configured to model a different portion of the complex system, the method further comprising: for each agent model of the plurality of agent models, determining an intersecting rule set between the agent rule bases of said each agent model and any of those other agent models in the plurality of agent model(s), wherein the agent rule base of each of the plurality of agent model(s) intersects with at least one other agent rule base of another of the plurality of agent model(s); and for each agent model of the plurality of agent models, merging said each agent model with each of those agent models in the plurality of agent models determined to intersect with said each agent model to form an intermediate fused or integrated agent model based on combining those one or more layer(s), AS node(s), and/or AU(s) of each of the plurality of agent models that intersect; merging each of the intermediate fused or integrated agent models to form an fusion agent model; and updating the fusion agent model based on one or more validation and training labelled datasets associated with each of the plurality of agent models until the integrated model is validly trained.

As a further option, the computer implemented method of any of the first, second and/or third aspects, wherein determining an intersecting rule set between at least the first trained agent model and second trained agent model further includes determining a compatibility score between at least the first trained agent model and the second trained agent model, and indicating those models of at least the first and second trained agent models to be merged when the compatibility score is above a predetermined threshold.

Optionally, the computer implemented method of any of the first, second and/or third aspects, wherein calculating the compatibility score comprises determining whether one or more semantic relationships exist between at least the first trained model and at least the second trained model.

As an option, the computer implemented method of any of the first, second and/or third aspects, wherein determining whether one or more sematic relationships exist further comprises forming a semantic network between at least the first trained model and the second trained model, wherein interconnections in the semantic network exist when one or more entities associated with the first trained model are connected, correlate or have a relationship with one or more entities associated with the second trained model.

As another option, the computer implemented method of any of the first, second and/or third aspects, the steps of determining an intersection rule set and merging at least the first trained agent model and at least the second trained agent model further comprising: determining one or more areas of similarity between agent state networks of at least the first trained agent model and second trained agent model; comparing, based on each area of similarity, the AS rule sets of the AS nodes in the area of similarity between at least the first trained agent model and the second trained agent model; and merging, based on the comparison of each area of similarity, the corresponding AS nodes and interconnections between the layers of at least the first and second trained models.

Optionally, the computer implemented method of any of the first, second and/or third aspects, further comprising: determining, using a graph matching algorithm, the one or more areas of similarity between at least the first trained agent model and the second trained agent model; and merging the corresponding AS nodes and interconnections further comprising: concatenating, based on the determined areas of similarity, the corresponding sets of AS rules and AS node states of the at least first trained agent model and the second trained agent model; and applying a belief function to the concatenated set of AS rules and AS states.

As another option, the computer implemented method of any of the first, second and/or third aspects, further comprising: training each agent model (100) to model one or more portions of the complex system using a labelled training dataset comprising a plurality of labelled training data items corresponding to the one or more portions of the complex system, wherein interconnections between AS nodes are initially randomised, training each agent model (100) further comprising: receiving each labelled training data item from a source (110) and vectorising each received labelled training data item; processing, by at least one of the input, intermediate and output layer(s), each vectorised training data item by the corresponding AS node(s) (190), wherein the AS node(s) (190) are located in the same (150) or different layers (130, 160) and perform at least one of a plurality of functions; outputting, from the output layer, an output vector (170) for each labelled training data item in the labelled training dataset based on the processed vectorised training data item; and updating the AS node(s) of at least one of the input, intermediate layer(s) based on comparing each output vector with each corresponding labelled training data item.

As a further option, the computer implemented method of any of the first, second and/or third aspects, wherein: receiving and vectorising each labelled training data item further comprising receiving each labelled training data item and converting each labelled training data item into an input training data vector of a predetermined size, wherein the input training data vector includes feature elements associated with the training data item and two or more elements representing the label; processing each vectorised training data item further comprising: propagating one or more portions of each input training data vector to one or more AS(s) of the input layer, wherein each AS node uses a plurality of AUs to process the propagated corresponding one or more portions of each input training data vector for outputting an input AS node output vector; propagating each input AS node output vector from each of the AS node(s) of the input layer to correspondingly connected downstream AS node(s) of at least one of the intermediate and output layer(s), wherein each downstream AS node processes one or more propagated input AS node output vector(s) using the corresponding plurality of AUs and outputs a downstream AS node output vector(s); and iteratively propagating each downstream AS node output vector to correspondingly connected further downstream AS node(s) of at least one of the intermediate and output layer(s) for processing and outputting further downstream AS node output vector(s) until all of the AS node(s) of the output layer receive all of those downstream AS node output vector(s) from the corresponding connected AS nodes of said at least one intermediate layer; outputting, from the output layer, an output vector (170) for each labelled training data item further comprising: outputting an output AS node vector corresponding to each labelled training data item based on processing, by the one or more AS node(s) of the output layer, those received downstream AS node output vector(s) associated with said each labelled training data item; interpreting or classifying the output AS node vector to form a predicted label associated with the output AS node vector; updating the AS node(s) of at least one of the input, intermediate and output layer(s) further comprising: evaluating, for each output AS node vector corresponding to each labelled training data item, an indication of an error between the predicted label associated with the output AS node vector and the label associated with the corresponding labelled training data item using a cost function; and performing a minor mutation of the agent network state and agent rule base based on the indication of the error; repeating the receiving and vectorising, processing, outputting and updating steps for each labelled training data item of the labelled training data set until one or more of: a minimum error rate is achieved for all the labelled training data items of the labelled training dataset; a set number of training epoch cycles of the labelled training dataset is achieved; a set number of training cycles for each labelled training data item is achieved; and in response to a set number of epoch cycles being met and an error rate for all the labelled training data items being greater than the minimum error rate, then performing a major mutation of the agent network state and agent rule base based on mutating the interconnection topology of the agent network state and/or one or more AS rules of the agent rule base and repeating the training steps of: receiving and vectorising, processing, outputting and updating steps for each labelled training data item of the labelled training data set.

Optionally, the computer implemented method of any of the first, second and/or third aspects, prior to processing one or more input vector(s), each AS node waits for all AS nodes connected to said each AS node to send the corresponding one of the one or more input vector(s), and process(es) said one or more input vector(s) once they all have been received. As another option, the computer implemented method of any of the first, second and/or third aspects, once an AS node sends the corresponding output vector towards one or more connected AS node(s), sending by said AS node the output vector to each one or more upstream AS node(s) connected to said AS node. As a further option, the computer implemented method of any of the first, second and/or third aspects, wherein said each upstream AS node reduces the threshold for outputting an output vector.

Optionally, the computer implemented method of any of the first, second and/or third aspects, wherein: the plurality of agents of each AS node includes a designated input agent for receiving vectors from one or more upstream AS nodes connected to said each AS node and a designated output agent for propagating an output vector to one or more downstream AS nodes connected to said each AS node; each agent of the plurality of agents includes a set of agent rules from an agent rule base, the set of agent rules being the same for each agent of the plurality of agents; each of the agents of the plurality of agents operates on identically sized vectors, the vectors of each of the plurality of agents defining a vector state space or AS node state space; iteratively processing the input agent vectors of each agent received from those other agents connected to said each agent until a maximum number of iterative cycles based on the agent rule set, an activation threshold value modified by an activation threshold function in each cycle, and a current activation value modified by a current activation value in each cycle; outputting an agent vector for input to one or more other agents connected to said each agent when the current activation value satisfies the activation threshold function; and modifying activation threshold function downward to current activation value when the activation value is less than the activation threshold.

As an option, the computer implemented method of any of the first, second and/or third aspects, wherein each agent of the plurality of agent(s) of an AS node has a local state vector. As another option, the computer implemented method of any of the first, second and/or third aspects, wherein a value of the local state vector is updated after an iteration of a cycle and/or is set based on a historical value.

As an option, the computer implemented method of any of the first, second and/or third aspects further comprising: processing, the vectorised data at the input layer (130) comprises: determining a firing threshold at the input AS node based on the received one-dimensional data; computing, at the input AS node in the input layer (130), a transformation of the one-dimensional input vector to a first vector of first size based on the firing threshold of the input AS node; and transmitting or propagating, from the input layer (130) to the one or more intermediate layer (150-1), the first vector to each agent of the plurality of AS nodes.

As an option, the computer implemented method of any of the first, second and/or third aspects, wherein vectorising the received data further comprises splicing the received data into a one-dimensional input vector based on one or more of: propagating the one-dimensional input vector to each AS node of the input layer; dividing the one-dimensional input vector into one or more portions, wherein each portion is propagated to a different AS node of the input layer; or applying a sliding window of a fixed length over the one-dimensional vector for propagating corresponding fixed length portions of the one-dimensional vector to a different AS node of the input layer.

As an option, the computer implemented method of any of the first, second and/or third aspects, wherein each AS node of the one or more intermediate and output layer(s) is coupled to a select/reduce function component configured for receiving each of the one or more output vectors from one or more upstream AS node(s) connected to said each AS node, wherein the select/reduce function component combines or transforms the received one or more output vectors into an input vector for input to said each AS node. As another option, the computer implemented method of any of the first, second and/or third aspects, wherein collating, using the S/R function, the first vector comprises: comparing, at an AS, a length of the received first vector with a input vector local to the AS, selecting, a sub-set of values that are common to the first vector, and reducing the selected sub-set of values with a single value.

In a fourth aspect, the present disclosure provides a fused or integrated model for modelling a complex system according to the computer-implemented method according to and/or as described in any of the first, second and/or third aspects, one or more features and/or steps therein, modifications thereof, combinations thereto, as herein described and/or as the application demands.

In a fifth aspect, the present disclosure provides a fusion or integrated model trained according to computer-implemented method according to and/or as described in any of the first, second, third and/or fourth aspects, one or more features and/or steps therein, modifications thereof, combinations thereto, as herein described and/or as the application demands.

In a sixth aspect, the present disclosure provides a computer-readable medium comprising data or instruction code which, when executed on a processor, causes the processor to perform the computer-implemented method according to and/or as described in any of the first, second, third, fourth, and/or fifth aspects, one or more features and/or steps therein, modifications thereof, combinations thereto, as herein described and/or as the application demands.

In a seventh aspect, the present disclosure provides an apparatus comprising a processor unit, a memory unit, a communications interface, the processor unit connected to the memory unit and communications interface, wherein the apparatus is adapted to perform the computer-implemented method according to and/or as described in any of the first, second, third, fourth, fifth, and/or sixth aspects, one or more features and/or steps therein, modifications thereof, combinations thereto, as herein described and/or as the application demands.

In an eighth aspect, the present disclosure provides a system for generating an integrated or fused model from at least two agent model(s) for modelling a complex system, each agent model comprising: a plurality of agent system node(s), wherein each of the AS node(s) comprise a plurality of agent units, AUs, and a set of AS rules governing the plurality of AUs, each AU of the plurality of AUs connected to at least one other AU of the plurality of AUs; an input layer comprising a set of AS nodes of the plurality of AS node(s); an output layer comprising at least one AS node of the plurality of AS node(s); and one or more intermediate layer(s), each of the intermediate layer(s) comprising another set of AS node(s) of the plurality of AS node(s); wherein each agent model is trained to model one or more portion(s) of the complex system using a corresponding labelled training dataset, said each agent model being adapted, during training, to form: an agent rule base comprising one or more sets of AS rules; and an agent network state comprising data representative of the interconnections between the AS nodes of the input, output and intermediate layer(s), wherein the agent rule base and agent network state are generated during training and configured for modelling said portion(s) of the complex system; the system comprising: a receiver module configured to receive data representative of the at least two agent model(s); a rule intersection module configured to determine an intersecting rule set between the agent rule bases of at least a first trained agent model and a second trained agent model; a merging module configured to merge said at least first and second trained agent models to form an integrated agent model based on combining those one or more layer(s), AS node(s), and/or AU(s) of the first and second trained agent models that correspond to the intersecting rule set; and an integrated agent update module configured to update the integrated agent model based on one or more validation and/or labelled training datasets associated with each of the at least first and second trained agent model(s) until the integrated model is validly trained.

As an option, the system according to the eighth aspect, wherein the system is further configured and/or adapted to implement the computer-implemented method, apparatus and/or systems according to any of the first, second, third, fourth, fifth, sixth, and/or seventh aspects, one or more features and/or steps therein, modifications thereof, combinations thereto, as herein described and/or as the application demands.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1A is a schematic diagram illustrating an example model integration system according to some embodiments of the invention;

FIG. 1B is a flow diagram illustrating an example model integration process according to some embodiments of the invention;

FIG. 2A is a schematic diagram illustrating an example agent model system corresponding to the agent models for model integration of FIGS. 1A and 1B according to some embodiments of the invention;

FIG. 2B is a flow diagram illustrating an example agent model training process according to some embodiments of the invention;

FIG. 2C is a schematic diagram illustrating an example select reduce function for use with the agent model of FIG. 2A according to some embodiments of the invention;

FIG. 3A is a schematic diagram illustrating an example agent system node for use with the agent model of FIGS. 1A and 2B according to some embodiments of the invention;

FIG. 3B is a flow diagram illustrating an example agent system node process for use with the agent system node of FIG. 3A according to some embodiments of the invention;

FIG. 4A is a schematic diagram illustrating an example set of agent models for model integration/fusion according to some embodiments of the invention;

FIG. 4B is a schematic diagram illustrating an example agent model system corresponding to the agent models for model integration/fusion according to some embodiments of the invention;

FIG. 4C is a flow diagram illustrating an example agent model training process for use in training agent models according to some embodiments of the invention;

FIG. 4D is a schematic diagram illustrating a flattening process in the agent model training process of FIG. 4C according to some embodiments of the invention;

FIG. 4E is a schematic diagram illustrating a splicing process in the agent model training process of FIG. 4C according to some embodiments of the invention;

FIG. 4F is a schematic diagram illustrating a agent system node in the agent model of FIG. 4B or agent model training process of FIG. 4C according to some embodiments of the invention;

FIG. 4G is a schematic diagram illustrating a S/R function process in the agent model of FIG. 4B or agent model training process of FIG. 4C according to some embodiments of the invention;

FIG. 4H is a schematic diagram illustrating an example AS network state in the agent model of FIG. 4B or agent model training process of FIG. 4C according to some embodiments of the invention;

FIG. 4I is a schematic diagram illustrating an example agent model evaluation component in the agent model of FIG. 4B or agent model training process of FIG. 4C according to some embodiments of the invention;

FIG. 5A is a schematic diagram illustrating an example fusion model system according to some embodiments of the invention;

FIG. 5B is a schematic diagram illustrating an example semantic model according to some embodiments of the invention;

FIG. 5C is a schematic diagram illustrating an example semantic model merge according to some embodiments of the invention;

FIG. 5D is a schematic diagram illustrating an example AS graph network merge according to some embodiments of the invention;

FIG. 6A is a flow diagram illustrating an example partial intersection model integration/fusion process according to some embodiments of the invention;

FIG. 6B is a flow diagram illustrating an example full intersection model integration/fusion process according to some embodiments of the invention;

FIG. 6C is a flow diagram illustrating an example model integration process for integrating multiple agent model(s) according to some embodiments of the invention;

FIG. 7A is a schematic diagram illustrating an example computer apparatus/device according to some embodiments of the invention; and

FIG. 7B is a schematic diagram illustrating an example model integration system according to some embodiments of the invention.

Common reference numerals area used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

The invention provides a system, apparatus and/or method for efficiently and accurately fusing or integrating multiple machine learning (ML) models or kernels, each ML model or kernel configured to model an aspect of a complex problem or system into a single fused or integrated model that models the complex problem or system. The term fusion model or integrated model may be used to describe the resulting model for modelling the complex system. Two or more of the ML models/kernels may represent the same data sets (i.e. two different solutions in relation to the same input data set) and/or related data sets (i.e. each solution is provided with a different but related data set). Each of the multiple kernels (or ML models) may be related to at least one other of the multiple kernels, such that the multiple kernels may be fused or integrated together to form an integrated/fusion kernel or ML model that models/solves an overall complex system or problem. Deep kernel or model integration may be performed to create a model that is concordant with each of the original kernels that comprise original datasets. The resulting model is called a fusion model or an integrated model.

Each of the ML models or kernels may be based on a type of ML algorithm that uses a series of interconnected multi-agent systems (MASs) where (a) rules within each of the MASs are optimised; and (b) functions between the MASs in the wider network are optimised. By employing the model fusion/integration algorithm as described herein for each ML model that may be used to solve one or more aspects of a complex problem/system, an enriched predictive fusion model (kernel) may be created based on merging each ML model/kernel. When each ML model/kernel is configured as a MAS, each ML model/kernel has a multi-agent structure that may be integrated deeply together to “grow” a fusion or integrated kernel/model (also known as an integrated/fusion artificial intelligence (AI), when employed in parallel. The process of fusing or integrating said multiple ML models/kernels to form a single integrated model/kernel is called “model integration” or “model fusion”.

In order to join or integrate different kernels/ML models together, they should be “compatible”, i.e. are associated with a similar and/or common relationships in relation to their datasets and/or operate to solve and/or perform as a similar or common complex problem. Thus, a process of joining different kernels to form an integrated/fusion model may be applied based on identifying those kernels most likely to be compatible, and integrating these kernels to form an integrated/fused kernel for solving the complex problem or system in which each kernel solves an aspect or characteristic of the complex problem. For example, each kernel may have a predictive ability and be related to each other based on their respective data sets by way of different features, e.g. for example blood data (data set 1 for kernel 1) and cell data (data set 2 for kernel 2), in which each of the kernels 1 and 2 may be used to predict a common system (e.g. predict diabetes) independently. The process of generating an integrated/fusion model may be to use the blood dataset and the cell data set together to form an integrated/fusion model that more accurately predicts diabetes based on either or both blood data set inputs and/or cell data set inputs and the like.

The simplest form of model integration or fusion is a linear model fusion and its derivatives. Linear model fusion may involve translating the outputs of one kernel into another. However, linear model fusion (or integration) may get more complex as the “internals” of the model are never exposed. Modulations in the in-to-out transit, such as weighted mean, could be applied in an optimisation process that result in a “model ensemble”. However, embodiments of the present invention represent a much deeper model fusion than simple linear fusions/integrations and modulations. As described in the various embodiments, a set of rules that govern each ML model's behaviour is presented that are semantically attributed to real-world system components. The fusion process optimises rule concordance into a single model, thereby creating a fusion/integrated model or kernel that is representative of its constituents.

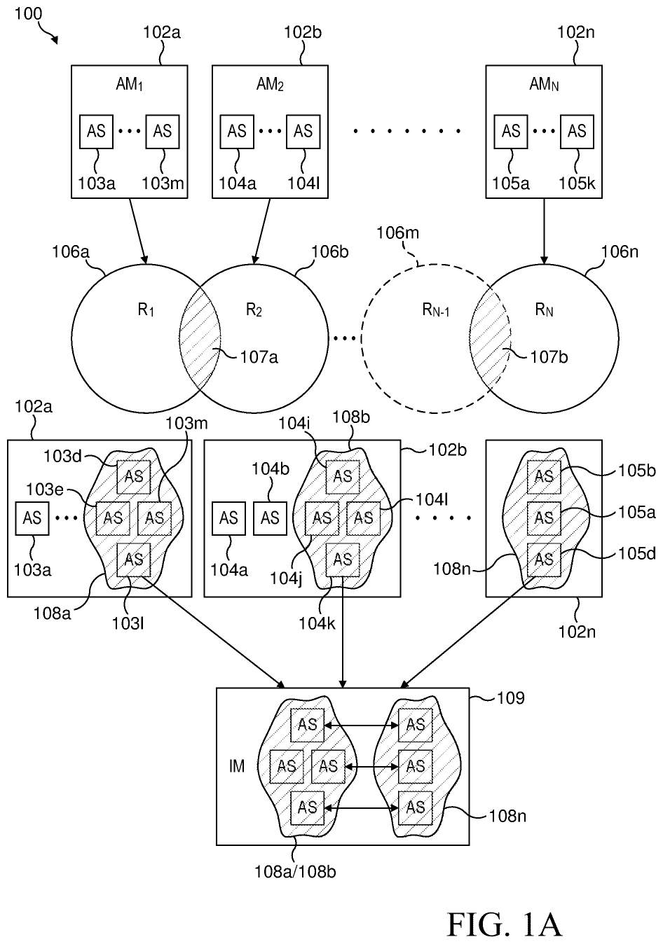

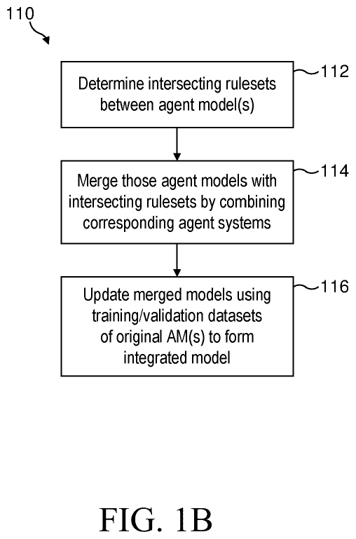

FIG. 1A is a schematic diagram illustrating an example model fusion or integration system 100 according to the invention. The model fusion system 100 is configured to generate a fusion model that models a complex system, in which one or more aspects of the complex system are modelled by at least two agent model(s) or a plurality of agent model(s) AM(s) 102a-102n. Each of the AMs 102a-102n are configured to model an aspect or characteristic of the complex system. Each of the AMs 102a-102n include a plurality interconnected agent system (AS) nodes 103a-103m, 104a-104l, 105a-105k, respectively. For an AM 102a, each AS node 103a of the plurality of AS nodes 103a-103m include a plurality of agent units (AUs) (not shown), and a set of AS rules governing the plurality of AUs. Each AU of the plurality of AUs is connected to at least one other AU of the plurality of AUs. For an AS node 103a, the corresponding plurality of AUs forms an interconnected AU network. Each of the AMs 102a-102n includes a plurality of layers in which each layer includes a set of AS nodes of the plurality of AS nodes 103a-103m, 104a-104l, 105a-105k, respectively. For each AM 102a, the plurality of layers of AS nodes 103a-103m include an input layer, one or more intermediate layers, and an output layer.

For example, an AM 102a may include an input layer having a set of AS nodes of the plurality of AS node(s) 103a-103m, an output layer having at least one AS node of the plurality of AS node(s) 103a-103m different to the set of AS nodes of the input layer, and one or more intermediate layer(s) between the input layer and output layer, where each of the intermediate layer(s) includes another set of AS node(s) of the plurality of AS node(s) 103a-103m different to the AS nodes of the input layer and output layer of the AM 102a. The AS node(s) 103a-103m of each of the layers are interconnected with one or more AS nodes 103a-103m of each of the other layers. For example, the set of AS nodes of the input layer are connected to another set of AS nodes of an intermediate layer, and the other set of AS nodes of the intermediate layer may be connected to a further set of AS nodes of another intermediate layer, if any, and so on, until the set of nodes of the last intermediate layer are connected to at least one AS node of the output layer.

Each of the AM(s) 102a-102n are trained to model one or more portion(s) or aspects of the complex system using a corresponding labelled training dataset. Each AM 102a being adapted, during training, to form: an agent rule base 106a (e.g. R₁) that includes one or more sets of AS rules; and an agent network state that includes data representative of the interconnections between the AS nodes 103a-103m of the input, output and intermediate layer(s) of the AM 102a. The agent rule base 106a and agent network state are generated during training of the AM 102a and when validly trained are configured for modelling said portion(s) or aspect of the complex system that is being modelled. Each of the AMs 102a-102n may be, without limitation, for example trained to model a different aspect or portion of the complex system and so may be trained using different labelled training datasets associated with the corresponding aspect or portion of the complex system being modelled. Thus, once trained, each of the AMs 102a-102n have a corresponding rule base 106a-106n (e.g. R₁, R₂, . . . , R_{N-1}, R_{N}) and agent network state corresponding to the interconnections between the AS nodes of each of the AMs 102a-102n. It is noted, that the interconnections between the AS nodes of each of the AMs 102a-102n are most likely different. Similarly, each rule base 106a-106n may each include different sets of AS rules and the like. However, given that each AM 102a-102n are modelling an aspect and/or a portion of the same complex system that is being modelled, it is highly likely that one or more of the AMs 102a-102n are related to at least one other of the AMs 102a-102n. For example, the data representative of each of the labelled training datasets used to train each of the AMs 102a-102n may be related in some manner, i.e. there is a relationship of some sort or a semantic relationship, to one or more of the labelled training datasets of at least one other AM of the plurality of AMs 102a-102n.

In this example, AM 102a (e.g. AM1) is illustrated as being related in some manner to AM 102b (e.g. AM2) such that there is an intersection of the rule bases 106a and 106b, meaning that there is a set of rules 107a that are common to both AM 102a and 102b. The relationship may be a semantic relationship and/or a causal relationship of some sort between the different AMs 102a and 102b. Similarly, AM 102n (e.g. AMN) is illustrated as being related in another AM of the plurality of AMs 102a-102n such that there is an intersection of the rule bases 106n and 106m, meaning that there is another set of rules 107b that are common to both AM 102n and the other AM. Furthermore, several other AMs of the plurality of AMs 102a-102n may have an intersection of rule bases between them, where these other AMs may also intersect with AMs 102a, 102b and/or 102n and the like, such that the rule bases 106a-106n of each of the corresponding AMs 102a-102n intersect with the rule base of at least one other AM of the plurality of AMs 102a-102n.

The complex system achieves integration of at least two AMs 102a-102b to produce an integrated AM 109 by combining an intersecting rule set between the agent rule bases of at least a first trained AM 102a and a second trained AM 102b. This is achieved by determining the intersecting rule set between the agent rule bases of the at least one of the first 102a and second AM 102b trained AM, and merging at least one of the first 102a and second 102b trained AMs, based on the determined intersecting rule set, to form an integrated AM 109. The at least first 102a and second 102b trained AMs are merged by combining one or more layer(s) 108; AS node(s) 103a, 104a; and/or AU(s) 302a of the first 102a and second 102b trained AMs that correspond to the intersecting rule set. The integrated AM is updated based on one or more validation and training labelled datasets, associated with each of the at least first 102a and second 102b trained AMs, until the integrated model 109 is validly trained.

As illustrated in FIG. 1A, the model fusion or integration system 100 is configured or operates to merge or combine the AMs 102a-102n to form a fusion or integrated agent model 109, which includes a set of AS nodes derived by merging and/or combining the AS node structures 108a-108n of each of the AMs 102a-102n that are associated with the intersecting rule sets 107a-107n of the rule bases 106a-106n of the corresponding AMs 102a-102n. This is achieved by the model fusion system performing a determination of each of the plurality or multiple intersecting rule sets 107a-107b between the agent rule bases 106a-106n of the AMs 102a-102n. This may include identifying the AS node structures 108a-108n of each of the AMs 102a-102n that correspond to the intersecting rule sets 107a-107b of the agent rule bases 106a-106n. Thus, the identified AS node structures 108a-108n of those AMs 102a-102n that have intersecting rule sets 107a-107b with other AMs 102a-102n may be merged to form a fusion or fused agent model 109. This may be achieved based on combining/merging the identified AS node structures 108a-108n, each of which may include one or more layer(s), AS node(s), and/or AU(s) associated with the intersecting rule set 107a-107b, from each AM 102a-102n with the corresponding identified AS nodes structures 108a-108n of each of the other AMs 102a-102n that correspond to the intersecting rule sets 107a-107b. For example, the rule base 106a of AM 102a has an intersecting set of rules 107a with the rule base 106b of AM 102b. This intersecting set of rules 107a corresponds to AS node structure 108a for AM 102a and AS node structure 108b for AM 102b. AS node structure 108a of AM 102a includes a first set of interconnecting AS nodes 103d, 103e, 1031 and 103m. AS node structure 108b of AM 102b includes a second set of interconnecting AS nodes 104i, 104j, 104k and 104l. Similarly, the rule base 106n of AM 102n has an intersecting set of rules 107b with the rule base 106m of another AM. AS node structure 108n of AM 102n includes a third interconnecting set of AS nodes 105a, 105b and 105d. Thus, the first and second set of interconnecting AS nodes 108a-108b are merged or combined together along with the third set of interconnecting AS nodes 108n, where the input layer, output layer and one or more intermediate layers of the fusion model 109 each includes a corresponding set of AS nodes from the merged sets of interconnecting AS nodes 108a-108n.

Although the fusion model 109 may be used, the fused agent model 109 (or fusion model 109) is updated based on one or more validation and training labelled datasets associated with each of the agent model(s) 102a-102n. This is to ensure the interconnections and weights associated with the AS nodes, AUs of each AS node, of the fusion model 109 are further optimised. Thus, the updates are performed on the fusion model 109 until the fused agent model 109 is validly trained. Once validly trained, the fused agent model becomes a trained fusion agent model 109 or fusion model.

In the medical field, model fusion/integration system 100 may be particularly useful in generating a highly accurate fusion model based on merging at least two or more agent models 102a-102n into an integrated model for modelling a complex system such as, without limitation, one from the group of liver disease diagnostics; prostate cancer diagnostics; bone fracture diagnostics; and/or any other disease diagnostics and/or state of a subject diagnostics; or more generally, any (set of) problem(s) that together form a multimodal behaviour, which can be modelled via a (set of) pattern recogniser(s). The AMs 102a-102n may be configured to detect and/or predict/classify whether a subject has a disease and/or detect/predict/classify the state of a portion of the subject or the state of the subject. Although the model fusion/integration system 100 and resulting fusion model 109 are described for use in modelling complex systems and/or solving complex problems in the medical or clinical fields, this is by way of example only and the invention is not so limited, it is to be appreciated by the person skilled in the art that the model fusion/integration system 100 may be applied to any other type of field such as, without limitation, for example, data informatics, biomedical and/or chem(o)informatics; oil/gas detection; geographical fields; and/or any other technical field in which complex system may be modelled for detecting, predicting and/or classifying and the like.

For example, the complex system to be modelled may be based on detecting a disease or state of a subject from one or more images of the subject. A fusion agent model 109 may be obtained that is configured as described above for modelling the detection of the disease or state of the subject when one or more images of the subject are input to the fusion agent model. The fusion agent model may be derived from at least two agent model(s) 102a-102b in which each AM 102a and 102b may be trained to model the detection of the disease or state of a subject from a different imaging source. Each labelled training data set may include a plurality of images of subjects that have been annotated and/or labelled to indicate whether the disease or state of the subject is present or not. Each of the plurality of images in the labelled training data set may be annotated and/or labelled to indicate whether portions of the images are associated with a tumour, disease or state of the subject, whether the tumour, the locations of the portions of the images associated with the tumour, disease or state of the subject, and/or whether the tumour, disease or state of the subject is present or not. For example, the labelled training data set for a first AM 102a may be based on images produced from a medical imaging system of a first type or a first format/resolution and the like, and the labelled training data set for a second AM 102b may be based on images produced from a medical imaging system of a second type or a second format/resolution and the like. For example, the manufacturer may determine the type of the medical imaging system, thus the medical imaging system of the first type may be manufactured by a first manufacturer, and the medical imaging system of the second type may be manufactured by a second manufacturer different to the first manufacturer.

Although the first and second medical imaging system may output a similar images of a subject in relation to assisting in detecting a tumour, disease and/or state of the subject, there may be differences in the formatting of the images from different manufacturer equipment such that these may cause inaccuracies in any model that is jointly trained on both sets of images. Although AMs 102a and 102b may be trained on particular types of labelled training datasets, where the outputs could be combined/weighted to provide a joint output, this results in a sub-optimally designed model of the complex system, which may discount or miss essential information associated with how each AM 102a-102b are related to the other. Thus, the fusion system 100 takes into account the related structures of each of the AMs 102a-102b, where each AM 102a and 102b is trained specifically on one type of labelled training data set of images, such that the resulting fused model or fusion model 109 is jointly optimised. This is achieved by merging the AS node structures 108a-108b of AMs 102a-102b together to form fusion model 109, which is further optimised by jointly training with both labelled training data sets.

In the above example, the complex system to be modelled may be detection of prostate cancer of a subject, which is modelled by a plurality of prostate cancer detection agent models 102a-102n, where each prostate cancer detection agent model 102a is trained using a labelled training dataset including a plurality of labelled training data images of subjects in relation to detecting or recognising prostate cancer tumours from said labelled training data images. Each labelled training data image may be annotated with one or more labels indicating whether the subject associated with that image has prostate cancer tumours, and/or, if the subject has prostate cancer, then further labels indicating data representative of the position or location of the prostate cancer tumour and the like. Each prostate cancer detection agent model may use a labelled training dataset based on images output from the same type of imaging system, which is different to the imaging systems used in each of the other prostate cancer detection agent models of the plurality of prostate cancer agent models 102a-102n. For example, each imaging system may be, without limitation, for example a particular magnetic resonance imaging (MRI) system made by a different or particular manufacturer, or any other type of imaging system used for imaging the subject as the application demands.

In another example, the complex system may be bone fracture detection of a subject that is modelled by a plurality of bone fracture detection agent models 102a-102n. Each bone fracture detection agent model may be trained using a labelled training dataset including a plurality of labelled training data images of subjects in relation to detecting or recognising bone fractures from said labelled training data images. Each bone fracture detection agent model may use a labelled training dataset based on images output from the same type of imaging system. Each labelled training data item is annotated or labelled as to whether or not said subject of the plurality of subjects has a bone fracture and/or a predicted location of where the bone fracture may be located within the image of the subject. Each bone fracture detection agent model is trained in relation to images associated with different imaging systems. Thus, the model fusion system 100 may be used to generate a bone fracture fusion detection model for use in modelling a complex system of detecting a bone fracture and/or assisting in bone fracture diagnosis and the like. As an example, each imaging system may be, without limitation, for example a particular model X-ray scanning system by a particular and/or different manufacturer and the like and/or as the application demands.

FIG. 1B is a flow diagram illustrating a model fusion or integration process 110 that may be used by the model fusion system 100 of FIG. 1A according to the invention. For simplicity and by way of example only, the reference numerals of FIG. 1A may be re-used for similar and/or the same components, features and the like. This should be considered exemplary and not necessarily limiting. Additionally, each of the steps of the model fusion or integration process 110 illustrated in FIG. 1B may include further steps, which may be a one directional flow, but may also be iterative in nature. It is assumed that the plurality of AMs 102a-102n have been trained on their respective labelled training data sets and that each AM of the plurality of AMs 102a-102n models one or more aspects or portions of the complex system that is to be modelled by the resulting fusion model 109. It is also assumed that each of the AMs 102a-102n have a relationship link (e.g. semantic relationship) in some manner to one or more other AMs of the plurality of AMs 102a-102n, whereby each AM 102a-102n has a rule base 106a-106n in which a set of rules of the rule base 106a-106m intersects with a set of rules of another rule base 106a-106n. It may also be assumed that the rule base 106a of each of the trained AMs 102a-102n intersect with one or more other rule bases 106a-106n of another one or more trained AMs 102a-102n, and that all the AMs 102a-102n with intersecting rule bases 107a-107b are linked or connected together via the rule base intersections 107a-107b such that there are no mutually exclusive groups of AMs with intersecting rule bases. The model fusion or integration process 110 may include the following steps of:

In step 112, the process 110 determines a set of intersecting rules between all the AMs 102a-102n. For example, this may include determining based on the rule bases 106a-106n a set of intersecting rules between at least two AMs 102a and 102b. For example, two AMs 102a-102b may each be trained to detect/diagnose prostate cancer based on MM scan of prostate or detect a bone fracture on an X-ray scan. The process 110 may determine a set of intersecting rules by analysing a set of rules from rule base 106a generated by training the individual AM 102a to detect prostate cancer or a bone fracture based on a first type of imaging system, and the set of rules from rule base 106b generated by training the individual AM 102b to also detect prostate cancer or a bone fracture based on a second type of imaging system.

As an example, the set of intersecting rules may be determined using a compatibility score between at least a first trained AM 102a and a second trained AM 102b. Based on the determined compatibility score, for example when the compatibility score is above a predetermined threshold (for example a AS node error threshold, a AS node firing/activation threshold, one of more layer excitation/activation threshold etc.,), then at least the first trained AM 102a is merged with the second trained AM 102b. Additionally and/or alternatively, the compatibility score may be a similarity score, and is determined using a matching algorithm, for example, a node graph matching algorithm/function (XNX). The XNX matching algorithm is configured to consider graphs or subgraphs of interconnected AS nodes of at least two AMs 102a-102b as arguments and develops a similarity measure between 0 and 1 in relation to the two AMs 102a-102b, for example where 0 denotes complete non-similarity and 1 denotes complete similarity. This may be used to determine the AS node structures of each AM 102a-102b that are similar or have complete similarity and those that are not similar, or have complete non-similarity. Alternatively or additionally, the compatibility/similarity score may be a calculated score, based on a simple comparison matrix, for example comparing a number of AS nodes at a stage in training each AM 102a-102n.

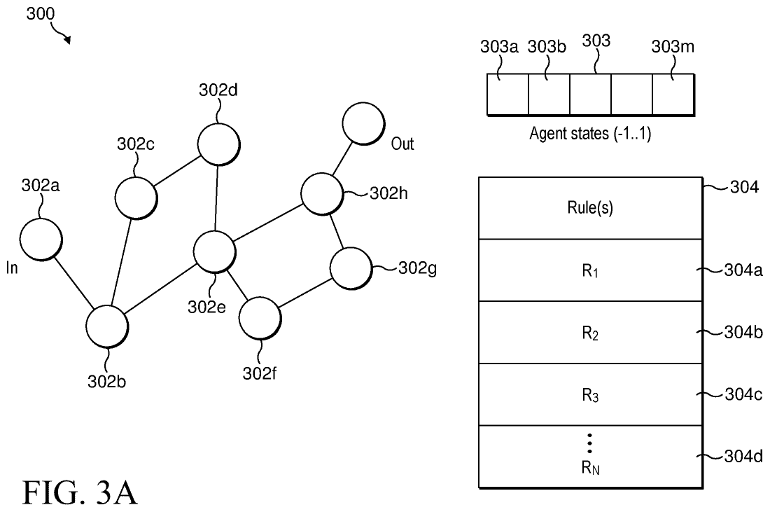

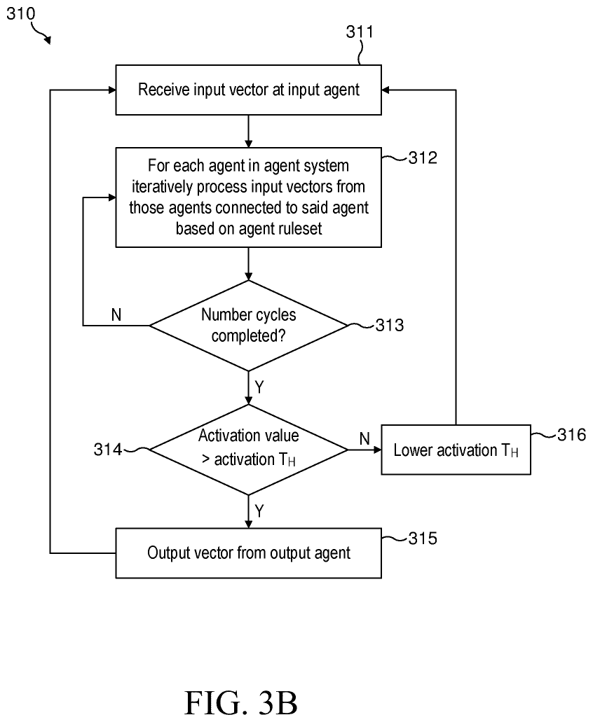

In a further alternative, the compatibility/similarity score may be calculated, based on one or more semantic relationships that exists between at least the first trained AM 102a and at least the second trained AM 102b. The semantic relationship is determined based on a nature of interconnections that exist in a semantic network (between the at least one first trained AM 102a and the second trained AM 102b). For example, in a similarity network one or more entities (for example concepts) associated with the first trained AM 102a is connected/correlated or has a relationship (direct/indirect) with one or more entities associated with the second trained AM 102b. By comparing the association(s), for example by comparing the set of rules governing the AS node 302a (e.g. shown in FIG. 3A) of the first trained AM 102a and the second trained AM 102b, in areas of the similarity network, a determination is made and the compatibility/similarity score is calculated.

In step 114, based on determination of the set of intersecting rules, the process 110 merges the at least two AMs 102a-102b associated with the determined set of intersecting rules. The at least two trained AMs (for example, the first 102a and second 102b trained AMs) are merged by combining their respective AS nodes 103a-103m and 104a-104l associated with the intersecting set of rules to form a fused and/or integrated AS node 108a/108b of the fusion AM model 109. For example, if a set of rules governing the AS node 103a of the first trained AM 102a (e.g. where the first trained AM 102a is trained to detect prostate cancer from images acquired from an MRI machine made by a first manufacturer) is similar to the set of rules governing the AS node 104a of the second trained AM 102b (e.g. where the second trained AM 102b is trained to detect prostate cancer from images acquired from an MRI machine made by a second manufacturer), the system merges the AS nodes 103a and 104a of the first 102a and second 102b trained AMs to form an integrated AS node of AS node structure 108a/108b associated with the intersecting/overlapping rule sets 107a. Each integrated AS node of the integrated AS node structure 108a/108b may be created by merging the entire AS node 103a and AS node 104a or by combining at least part of the AS node 103a and AS node 104a. However, the degree to which the AS nodes may be merged may depend on the underlying rule sets of the AUs associated with each AS node and/or factors/parameters that may be user driven.

As an example, merging the respective AS nodes, that is the first AS node 103a of the first trained AM 102a with first AS node 104a of the second trained AM 102b may be based on the determined area of similarity, for example similarity between the semantic networks, or similarity of interconnections between one or more layers of the first 102a and/or second 102b trained AMs. Merging the respective AS nodes 103a, 104a may further comprise concatenating set of rules and states governing the AS node 103a of first trained AM 102a with that of AS node 104a of second trained AM 102b.

Additionally and/or alternatively, a belief function may be applied to the concatenated set of rules and states governing the integrated AS node of the AS node structure 108a, 108b of the resulting fusion/integrated AM 109. The belief function may be any function that takes two n-length vectors and assigns a “belief” to each. Based on the belief, the function outputs a new n-length vector that could be identical or different to one or both the inputs (for example, based on the average per-locus). However, the application of the belief function may be user driven.

In step 116, after merging/integrating each of the AM 102a-102n and/or the AS node structures 108a-108n associated with intersecting rule sets 107a-107b to create a merged/integrated AS node structure 108a/108b/108n of the fusion model 109, the process 110 may then update the integrated AM 109. In other words, the integrated AM 109 may be retrained using training/validation datasets used to train the original AMs 102a-102n (for example, at least one of the first and/or second trained AM 102a-102b of the plurality of AMs 102a-102n). Here the training/validating dataset is a labelled dataset and the integrated model 109 is retrained until the accuracy of the result is at least close to or on par with the accuracy of first 103a and/or second 103b trained, or is above a predetermined threshold (as described herein).

In an example, let a data set D_1 be trained on a AM 102a creating an agent rule base R_(M1,D1) over state space S_(M1,D1). According to embodiments of the present invention, MI is the process by which D_2, a related data set in the same complex system that is being modelled, can be modelled into M_2 and then merged with M_1 with the help of a semantic network S which may bridge the gap between the abstract metamodel and the real world, for example, a simple semantic network that describes a tumour, which is part of tissue and is composed of cells. Cells divide, which is a behaviour that is manifested as tumour growth. If there is sufficient depth in training data, an optimised multi-agent model will develop rules and states where each of these concepts are represented within the network. The basic foundation of the semantic model may be a list of entities (real world or concepts); a list of connections between those entities that may define non-physical connectivity (for example, the computation of one concept includes/consumes the computation of another concept) and physical connectivity (for example, an entity is part of or entirely consumed physically by another entity). However, in other examples, this may extend and become more specialised for each field of study.

In this example, the resulting fusion model or integrated model (IM) 109 is configured to: receive D_1-like input and create predictions like D_1 even in the absence M_2 inputs; receive D_2-like input and create predictions like D_2 even in the absence M_1 inputs; predict the values missing inputs given an outcome from any input; and predict outcomes when both D_1 and D_2-like inputs are present, even if the outcomes are conflicting. For example, a real-world system, such as the behaviour of a complex disease, may be captured in data silos, e.g. imaging of a tumour and blood factors that indicate the severity of the disease. A model may be built on each data silo. AM 102a and AM 102b may therefore be independent models that have predictive power, however they represent two different (perhaps related) aspects of the system. The process of model fusion tries to find a computational solution that respects the behaviour of each model constituent to create a single more representative model.

In another example, assume AM 102a has agent rules R_1 106a and AM102b has agent rules R_2 106b. There may be three possibilities for integration: no Intersection is performed because R_1 106a and R_2 106b have absolutely no intersection 107a according to their respective semantic models. In this scenario, there may be two choices for integration, such as the AM 102a-b may be run independently with no connectivity; and/or the models AM 102a-b may co-exist in the same IM 109 with independent state spaces but with a shared topology. Alternatively, a partial Intersection may be performed because R_1 106a and R_2 106b may have some intersection according to their semantic models. In this scenario, AM 102a-b may co-exist in the same IM 109 and therefore the requirements are: finding the intersecting rules 107a and optimising any conflict resolution between the intersecting rules 107a. Yet further alternate could be to perform a full Intersection when R_1 106a and R_2 106b may have complete intersection 107a according to their semantic models. In other words, they are competing models which should co-exist in the same IM 109 and therefore the requirements are to: optimise a conflict resolution between the rules 106a and 106b.

In another example, an XNX Matching Algorithm may be used in the context of IM 109. This may be any function that takes two graphs or subgraphs as arguments and develops a similarity measure between 0 and 1 where 0 denotes complete non-similarity and 1 denotes complete similarity. Radius of the similarly arc may be taken into consideration, i.e. the maximum radius to search for subgraph similarity. This function may be used to decide how to merge the two graphs. In another example, a function that defines how different nodes from different graphs may be merged with respect to: the corresponding AS states in each node, and the up and down connections in their respective graphs.

In another example, a belief function that takes two n-length vectors and assigns a “belief” to each may be used to arrive at IM 109. Based on the belief, a new n-length vector may be outputted which could be identical to one of the inputs or a mixture of the two (e.g. average per-locus).

In another example, mature kernels AM 102a and 102b may be deeply integrated, assuming a high degree of individual accuracy. The steps may be as follows: search for areas network similarity between 102a and 102b using the XNX graph matching algorithm; for each area of similarity above a predefined threshold—if there is an exact match, then superimpose AS state and rules, i.e. states and rules are concatenated and apply a belief function; else if there is a partial match above the threshold, then replace the area of similarity using a node (graph) merge function and apply a belief function; and rerun the ML algorithm to re-optimise the parameters. Alternatively, for each AS node the AS state and rules are superimposed and concatenated and a belief function is applied. The final IM 109 must fulfil a predefined maximum error with respect to all training sets as well as validation sets.

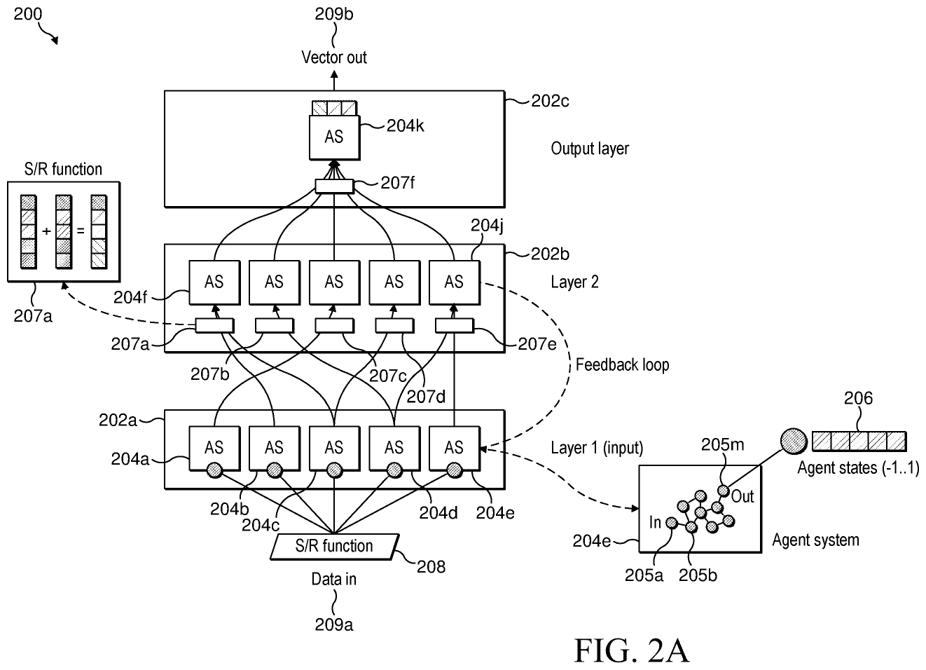

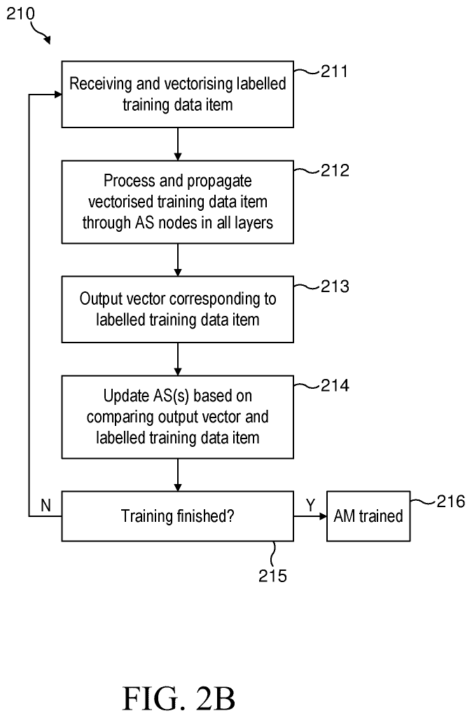

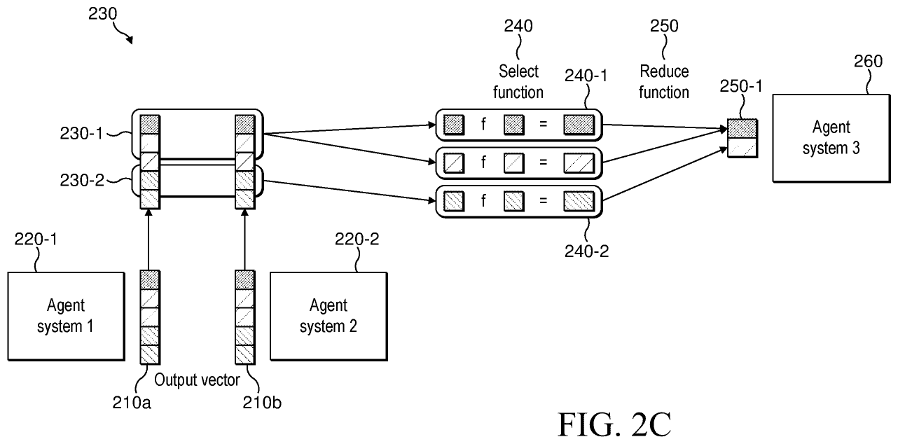

FIG. 2A, is a schematic diagram illustrating training of an example AM 102a that may be used for model integration with one or more other trained AMs 102a-102n (as shown in FIGS. 1a and 1b), according to some embodiments of the invention. In FIG. 2A, each AM 102a of a plurality of AM 102a-102n may include a plurality of layers 202a-202c, for example, an input layer 202a, an intermediate layer 202b, and an output layer 202c. The input layer 202a is connected to the intermediate layer 202b, which is connected to the output layer 202c. However, each layer 202a-202c may also connect to a previous layer, i.e. a feedback loop may exist between the layers 202a-202c. Each of the input layers and intermediate layers 202a-202b of the plurality of layers 202a-202c comprises a plurality of AS nodes 204a-204e or 204f-204j, respectively. The output layer 202c may include at least one AS node 204k. Each of the AS nodes 204a-204j may include a plurality of AU 205a-205m governed by a set of rules and/or agent states 206 as described herein with reference to FIGS. 1a to 7b.

Briefly, in FIG. 2A, when an input data source 209a receives labelled training data items, it vectorises the received data items for the input layer 202a, for example by using an assimilation and splicing function. The vectorised training data items are propagated from the input layer 202a through to the intermediate layer 202b and finally to the output layer 202c (downstream propagation), via a plurality of select/reduce functions (S/R function) 208, 207a-207e, 207f (an example of the S/R function is shown for AS node 204f as S/R function 207a) and 207f for the output layer 202c.

The output layer 202c generates an output vector 209b that, during training, may or may not correspond to each of the corresponding labelled training data items. The generated output vector 209b is compared with the corresponding labelled training data item, and based on the result of the comparison, at least one of the AS nodes 204a-204k of the input/intermediate/output layer 202a-202c is updated, for example the firing threshold of the AN node is modified, interconnections between AS nodes is adapted, and/or the interconnections between the individual AUs 205a-205m within, without limitation, for example an AS node 204e is modified (upstream propagation).

Based on the updated values, the AS nodes 204a-204k located in the same or different layers 202a-202c and may perform at least one of a plurality of functions based on the training/retraining of the labelled training data item and/or the feedback loop. For the purposes of the present invention, each training data item of the received labelled training data item iterates through each of the AM 102a layers, and/or the integrated model 109, at least once thereby setting certain training/model parameters such as: least a number of repetitions, certain error threshold for each repetition, and/or a consistency of error value. These training/model parameters may be user defined or may change based on the model type. Therefore, these training/model parameters should not be considered as binding on either the training of each of the AMs 102a-102n, using labelled training data items, and/or on the integrated model 109, that is created by merging/integrating at least two trained AMs 102a-102b of the plurality of AMs 102a-102n.

FIG. 2B is a flow diagram illustrating an example AM training process 210 for training an AM 200 as described with reference to FIG. 2A, according to some embodiments of the invention. Each of the AMs 102a-102n may be individually trained based on different labelled training data items in relation to training each of the AMs 102a-102n for modelling an aspect of a complex system. Thus, each of the labelled training data items for each AM will be focussed on assisting in training that AM to model its corresponding aspect of the complex system that it is modelling. Once they are all trained, the AMs 102a-102n may be integrated and merged to form a fusion model 109 that more accurately models the entire complex system as described herein. Thus, for each AM 202, the AM training process 210 may include the following steps of:

In step 210, the AM 200 receives and vectorises labelled training data item. After receiving the labelled training data items from the source, the received data is prepared/pre-processed by the assimilating and splicing function. However, other pre-processing techniques known to the person skilled in the art may be used to vectorise the received labelled training data items. In some embodiments, all of the vectorised training data items are propagated downstream at the same time. For example, one or more portions of each vectorised training data input is propagated downstream.

Assimilation of the received data may include, without limitation, for example normalisation of each of the labelled training data items and converting each of the labelled training data items into a corresponding input training data vector, of a predetermined size. The normalised input training data vector includes feature elements associated with the labelled training data item with two or more elements representing the single label. For example, an input labelled training data may contain certain physical human features, for example, height, weight, limb and torso dimensions. A single label for Gender could also be used in the set of vectors so that a single vector (containing a single data row), post-normalisation could represent not only the body features but also a gender associated with the training data set. For example, data vector [0.3, 0.1, 0.7, 0.3, 1, 0] wherein [1, 0] at the end may represent the label [Male, Female] whilst [0.3, 0.1, 0.7, 0.3] may represent elements of physical human features.

The normalised input training data vector is fed into a topology and nested (e.g. vectors may be nested, without limitation, for example images that have a Red/Green/Blue value for every pixel may be nested). The nested data is converted into a one-dimensional vector (also known as flattening) and spliced based on a topology/configuration of the AM 200 that is to be trained. The splicing function is based on, without limitation, for example the predetermined pattern based on the topology/configuration of the AM 200. For example, the vector may be fed into the topology and depending on the source data type, the vector could be arbitrarily nested. The example above for a simple CSV file is a 1-dimensional array. For images that may have a Red/Green/Blue value for every pixel it may be nested in the following configuration: [[0.32,0.56,0.77], [0.11,0,1], [0.44,0.87,0.4] . . . ]

In some examples, the nested data may not be spliced (for example, the normalised input data vector in its entirety is copied to every node embedded in the input layer 202a). In an alternate embodiment, the nested data may be equally spliced (wherein the normalised input data vector is divided equally (with leftover appended to the last splice) to every node embedded in the input layer 202a). Alternatively or additionally, the nested data may be equally spliced with data overlaps (wherein a sliding window is applied over the normalised input data vector and a fixed length copy is sent to every node embedded in the input layer 202a).

In step 211, the AM processes/propagates each vectorised training data item through the AS nodes 204a-204k located in various layers 202a-202c. For example, each vectorised training data item is propagated via one or more portions of the input layer 202a, and via the corresponding S/R functions 207a-207e into the intermediate layer(s) 202b. It is noted, that the state space of the vectorised training data item that is input to the AS nodes 204a-204e (the input layer 202a) of the input layer is fixed. However, during a training cycle, the size of each vectorised training data item is not necessarily the same or fixed, and in order to mitigate this inequality in vector size, the S/R functions 208 and 207a-207f are used prior to input to each AS node 204a-204k of the AM 200 to fix the size of the input vectors. Although the S/R functions 208 and 207a-207k are illustrated as being connected to an AS node 204a-204k, this is by way of example only and the invention is not so limited, for example the skilled person would understand that the S/R functions 208 and 207a-207k may be located between each connected layer 202a-202c (for example, between the AS nodes 204a-204e of input layer 202a and the AS nodes 204f-204j of the intermediate layer 202b), and/or may be included in the functionality of each AS node 204a-204k and the like and/or as the application demands. FIG. 2C describes the S/R function in further detail.

In step 213, the output layer 202c produces an output vector 209b that corresponds to the vectorised labelled training data item. The output vector 209b, at an output AS node 204k, corresponds to each item of the labelled training data item or a group of items of the labelled training data item (steps 210 and 215 are repeated for each item of the labelled training data item). In other words, the system 200 performs downstream propagation for each item of the vectorised labelled training data item. As the output AS node 204k does not receive strength signals (for example values of the firing threshold) from the data propagated downstream, it does not itself possess a firing threshold. However, the output AS node 204k performs various computation of the output data and passes it on an interpreter function (not shown in FIG. 2B) that modulates the output vector (for example by converting a data vector [0.7,−0.9] to “YES/NO” based on a user defined threshold value (for example, ±0.5 threshold)). The interpreter function passes that data to an evaluator function (not shown in FIG. 2B that calculates a cost function, for example using a Euclidian distance, between the predicted label (output vector) and each item of the vectorised labelled training data item. As all values of the vectorised labelled training data items are restricted to −1≤x≤1 (as selected values of the vector falls within the state boundary range from −1≤x≤1), the evaluator function calculates an error value (for example a single binary number, 0 and/or 1) that is used by the AM 200 to learn/mutate. In some embodiments, the interpreter and/or function may be an artificial neural network (ANN).

In step 214, based on the output vector 209b and a comparison of the output vector 209a and the corresponding labelled training data item, the AS(s) 204a-204k may be updated. For example, the comparison of the output vector 209a and corresponding labelled training data item may be used to calculate an error value, where the calculated error value (obtained in step 213) is used to adjust/update one or more threshold values of at least one of the AS nodes 204a-204j in the input layer 202a and intermediate layers 202b. These may be updated using the feedback loop.

For example, the AM 200 that includes an AS network enters a learning/mutation phase, based on the calculated error value. However, before entering the mutations phase, in an example, the feedback loop may be an optional step of the algorithm and may be designed for sequential ML, for example time series data analysis or natural language processing, where there is a requirement to retain a certain memory capacity whilst the machine ingests the next data point (the next time series step, or next word in the sentence).

In another example, when an AS node triggers and produces an output vector, that vector is sent to downstream AS nodes and additionally sent to upstream AS nodes that did not cause that AS to fire in the first place (i.e. dormant nodes). When an upstream AS receives that message, it may modify an excitation level threshold upwards with function a_{τ,t=1}=min [a(a_{τ,t=0}),1] wherein the function a may be any mathematical function, e.g. sigmoid, straight line, step.

In a further example, the purpose of this loop may be to drive the learning optimisation process in such a way that certain sections of the network coordinate recognition of certain sets of co-related features and therefore cluster together in the network. When a single feature is recognised, it “eases” the recognition of co-related features to enhance the overall pattern recognition.

In another example, vector messages may propagate downstream through the network and coalesce at the output AS, where a single output vector may be generated. The output vector, unlike ANNs, need not be a vector that matches the label vector length. For example, for a binary recognition ANN where the outcome is “Yes” or “No” the expected output vectors may be [1,0]=YES or [0, −1]=NO. In other words, the output vector length may be 2, and each locus is dedicated to a single label. In this case, locus 1 may be dedicated to “Yes” and locus 2 may be dedicated to “No”.

In another example, an additional recognition step may be added after the output node produces the output vector. For example, if a binary vector is 10-loci long, an additional deterministic function may be applied to translate that 10-length vector into a Yes or No. Such a function itself could be another ML, an ANN, or a simple heuristic. The expansion of the vector length beyond what is required aids the MI process because it builds into the model a possibility that there are as yet unknown states that may need to be optimised.

In an example, the fitness evaluation is a measure of how far the prediction (post interpretation) is from the expected value given the feature set and labels. This may be determined by one of the many known distance measures, e.g. Euclidean distance. Since all values in all loci are restricted to −1≤x≤1, the measure can be normalised into the same bounds. The resulting value is treated like an error value.

The mutation phase comprises a minor and a major mutation phase. Both phases are triggered by a set of predetermined conditions, configured at the beginning of the training cycle. For example, the system may be configured to trigger a learning/mutation phase using a mutation type (minor/major) after a set number of evaluations have been carried out by the evaluator function. The trigger may also be based on a trajectory of the error curve (for example a sigmoid, a straight line, and/or step function), a probability of the mutation permanently disrupting the upstream and downstream propagation of the vectorised labelled training data items, and/or a probability of the mutation violating a current S/R requirement/configuration etc. The predetermined conditions may be user defined or may be as an ANN.

In an embodiment, a minor mutation refers to the error value used to modify the network, for example, the AM 200 may be trained for each data item of the vectored training data items until a set number iteration of each data item of the vectorised training data item is complete/achieved. Alternatively, the network may be modified until a minimum error value for each data item of the vectorised training data item is achieved. In another embodiment, a major mutation may refer to the network modified more abruptly, for example at the beginning/end of a certain configurable point (for example, after an epoch completion).

In an example, both minor and major mutations may be triggered by three configurable states that are set at the start of the execution of the ML. They may be set after a set number of evaluations have occurred, after the trajectory of the error curve plateaus or spikes (or some other configurable trigger), and/or a combination of the above. Additionally, there may be various minor and major mutations depending on the severity of the error value. Based on the error value, a selection of candidate mutations are selected and then further filtered for suitability. Suitability may be determined by a set of conditionals that are evaluated: based on: will the mutation permanently disrupt the flow of messaging up and down the network, or does the mutation violate S/R requirements that are already in place; optionally supply a stochastic value to determine whether or not to proceed. The vast majority of mutations will be a simple rule modification that modulates locus-specific signals—this simple rule is called Self Adjustment. However, during the execution of a vector flowing to the output node, a monitor may record the changes or deltas of the signal at each vector locus which may then be used to determine which nodes contribute most/least to each output locus. Based on this determination, the self adjustment rule may be modified to inflate or deflate the particular locus value.

In another example, the mutations themselves may not be limited to the following configuration, each of which may be modulated based on the severity of the error value: AS node 300 internal reconnectivity (including neighbourhood function mutations); S/R function mutations (swap out functions for others, modulate parameterisation of those functions); agent rule mutations (modulate parameterisation of those functions, delete, create, duplicate rules, shuffle rule order); agent state mutations (randomise state); agent mutations (modulate cycle number, rotate input/output nodes, activation threshold modulations); whole network reconnectivity (break/create connections).

In a further example, the mutation phase may represent the “learning” phase of the algorithm. However, in the validation phase, the kernel may be tested against a data set that it has not yet experienced. In this phase, mutation may be switched off.

In step 215, the system checks to determine if the training is completed. For example, it checks whether the learning/mutation phases have ended, signifying that the training is complete. For example, by determining that a minimum error rate based on the output and all corresponding vectorised labelled training data items has been achieved, and/or a set number of training epoch cycles of the labelled training data items has been achieved. Alternatively, if the system determines that an epoch cycle threshold is reached, and the error value/rate for all labelled training data items is determined to be greater than the minimum error rate, the AM 200 continues with training and proceeds to step 211. In this event, the training process 200 may perform in the learning/mutation phase a mutation, which perturbs the connections and/or thresholds of the AS nodes and/or input/intermediate/output layers and/or the AUs of each AS or random ASs and the like. For example AM 200 may perform a major mutation of the agent network and agent rules (for example, alter the interconnection topology of the agent network state and/or one or more AS rules of the agent rule base) in order to reduce the error rates.

The purpose of this loop is to drive the learning optimization process in such a way that certain sections of the network coordinate recognition of certain sets of co-related features and therefore cluster together in the network. When a single feature is recognized, it “eases” the recognition of co-related features to enhance the overall pattern recognition.

FIG. 2C is a schematic diagram illustrating an example S/R function 230 for use with the AM 200 as described with reference to FIGS. 2A and 2B, according to some embodiments of the invention. The S/R function 230 includes a select function 250 and a reduce function 240. These could be any set of logical/mathematical functions with a state boundary of between −1≤x≤1. It is noted that the output vectors of one or more AS nodes such as a first AS node 220-1 (e.g. Agent System 1) and a second AS node 220-2 (e.g. Agent System 2) of a layer (e.g. AS nodes 204a-204e of input layer 202a connect to one or more AS nodes 204f-204j of the intermediate layer 202b), may be input to the S/R function 230, which is configured to output an input vector to the AS node 260 to which it connects to (e.g. AS node 204f) (Agent System 3). In this example, the S/R function 230 receives the output vector 210a of a first AS node 220-1 (e.g. Agent System 1) and the output vector 210b of a second AS node 220-2 (e.g. Agent System 2). For example, with reference to FIG. 2A, the S/R function 200 may be the S/R function 207a in which the output vector 210a referred to above may be the output vector generated by AS node 204b of the input layer 202a, which connects to the S/R function 207a the outputs an input vector to AS node 204f of the intermediate layer 202b, as well, the output vector 210b referred to above may be the output vector generated by AS node 204c of the input layer 202a, which connects to the S/R function 207a of AS node 204f the intermediate layer 202b. Thus, the S/R function 230 takes at least two input vectors 210a and 210b and selects the corresponding vector elements 230-1 and 230-2 based on the select function 240, and reduces the selected vector elements 240-1 to 240-2 into a fixed input vector 250-1 for input to the agent node 260 (Agent System 3) for processing and the like.

As an example, the select function 240 may be configured to map across vectors (for example, selects values of the vector that fall within the state boundary range from −1≤x≤1). The reduce function 250 may be configured to resolve any conflict between the output vector being assigned to the same AS node, for example AS node 204f-j in the intermediate layer 202b. For example, for the input node the S/R function is configured to reduce the incoming vector size based on state size of the AS node in the input layer. In this example, if the input node state size is between 1 and 3, then the resulting vector size is a simple matrix. However, if the input node state size is >=4, then the resulting vector size is a function that is dependent on a distribution function that describes the distribution of the vector as if it were a data series. Alternatively of additionally, the state size of the distribution function may be limited to a bucket size. However, other means of reducing the size of the vector may be available to the person skilled in the art.

The S/R function 230 receives one or more output vector and combines or transforms the received one or more output vector into an input vector for each AS node 204f-j in the intermediate layer 202b. Alternatively or additionally, as an example, a collation function (for example, located at the interface between the input layer 202a and intermediate layer 202b) may be used to collate all data vectors outputted by the input layer 202a before processing begins at the intermediate layer 202b. For example, the collation function may require an S/R function. In an example, for every input vector locus: a source to target mapping is selected (the loci to select from incoming vectors (i.e. a list of indices); a “select” function is selected, that maps across the incoming vectors at the same locus and reduces that collection of states to a single state; and a “reduce” function is selected, that resolves multiple states writing to the same target state.

FIG. 3A is a schematic diagram illustrating an example agent system node 300 for use with the agent model of FIGS. 1a and 2a according to some embodiments of the invention. Referring to FIG. 3A, the AS node 300 includes a plurality of AUs 302a-302h. Although each AU 302a of the plurality of AUs 302a-302h is connected to at least one adjacent AU 302b-302h, the AUs 302a-302h behave independently of each other. Each designated AS node 300 has an input AU 302a (configured to receive the vectorised labelled training data item as an input vector or collated data vectors and the like), and an output AU 302h (configured to propagate downstream the vectorised labelled training data item or processed input vector/output vector). Each AU 302a is governed by the set of rules or rule set, wherein each rule set is identical for each AU 302a-302h. For example a rule set for a particular AU may be to set its state locus value based on an average of its neighbouring state locus values. However, connection to the adjacent AU 300b-302h is established when the AM 102a (shown in FIG. 1A) is first run (e.g., for training iteration) with the vectorised labelled training data items, and eventually change after every learning/training cycle. For example, as the calculated error improves/reduces the number of AUs required may be less because the system 100 is improving in accuracy. Each AU 302a is also primed with a vector 303 of states 303a-303m and a corresponding activation threshold, both fixed length during the training cycle and modified during the learning phase (for example using a sigmoid curve). Herein, the phrase “rule base”, “rule set” or “set of rules” may be interchangeably used.

For example, each AS node 300 has a designated input agent, A₀, 302a that receives messages (input vectors) from upstream (upstream could be other AS or the Initiation S/R). Each AS node 300 has a designated output agent, Aₙ₋₁, 302h that propagates messages (vectors) downstream (downstream could be other AS or the the final output S/R). Each of the AUs 302a-302h is primed with the same rule base 304 that include some interaction rules, Rₙ 304a-304d. Every AU 302a-h within a single AS node 300 is primed with the same rule base 304. Each AU 302a-h is primed with a vector 303 of states 303a-303m. The vector 303 of states 303a-303m has a fixed length during a single learning cycle. All AUs 302a-302h have identical length state vectors.

In the learning phase, the state space of the vector 303 of states 303a-303m for each of the AUs 302a-302h is either randomly allocated or allocated by some other heuristic. An activation threshold value, 0≤a_{τ}≤1 and a current activation value 0≤a≤1 are set or defined. An activation function that modifies a, e.g. on a sigmoid curve (configurable), is also set and/or defined. An activation threshold function that modifies a_{τ}, e.g. on a sigmoid curve (configurable), is also set or defined. A maximum number of cycles, c, is also set and/or defined. Put simply, each AU 302a-302h in an AS node 300 behaves independently of the whole and the behaviour (or the state) of the AU 302a-302h at any time step is a deterministic function of its previous states (memory) and the state of its neighbouring AU 302b-302h in (to a degree of freedom of 1 though it may expand in later iterations. This is called the neighbourhood function, N_{A}.

The agents or agent units (AUs) 302a-302h within an AS node 302 are governed by a set of rules 304, which are identical across all agents 302a-302h. However, for a particular agent a rule 304a may take as input the entire state space of neighbouring agents 302a-302h connected to it that are determined by a connectedness function between agents 302a-302h and a neighbourhood function that determines connectedness. States may not be inferred for non-neighbours during rule execution. However, a rule of the rule base/set 304 may tap into agent historical state memory but may only use, as its input, present and past state memory, in its calculations for time step t can only use states from time steps <t. A rule from the rule base 304 executed for a particular agent can only affect that agent's state. A rule and the states that it affects are tied to the data set that is being learned and its corresponding semantic network.

As an example, if all the state locus values of all neighbour AS nodes is 1, and the system calculates the average, and sets that as the new state locus 1 value. Each agent may have a state space composed of loci wherein each loci may have a value in the range −1<=0<=1. Each rule operates on defined loci. For example, at t=0, an average rule operating on Agent 2 takes the neighbouring and self values (0.1, −0.3, 1), calculates the average and replaces that new value (0.27) into that locus for t=1. Every rule is executed before this agent system can be timestamped as t=1.

FIG. 3B is a flow diagram illustrating an example agent system node process 310 for use with the agent system node of FIG. 3A according to some embodiments of the invention. The agent system node process 310 may include the following steps of: In step 311, the AS node 300 receives an input vector at the input agent. That is the AS node 300 receives the collated data vectors output by at least one layer, for example see FIG. 2A, the input layer 202a/intermediate layer 202b. In step 312, for each AU 302a-h in the AS node 300, iteratively process the input vectors from those agents connected to said each AU 302a-h based on the agent ruleset. For example, for each AU, this may include iteratively processing the received collated data vector(s) based on the rules set governing the AU and in-turn the AS node 300. In step 313, the input vector or the received collated data vector(s) is iteratively processed by each AU until, for example, a maximum number of cycles based on the agent rule set has been reached. In step 314, it is determined whether an activation threshold value exceeds an activation threshold function or activation threshold (e.g. activation Tx). When the activation threshold value exceeds the activation threshold function or activation threshold (e.g. activation Tx), in step 315, the AS node 300 outputs an agent vector (e.g. an output vector) that becomes an input to one or more other S/R function and/or an AS nodes. However, in step 313, if the activation threshold value does not exceed the activation threshold function or activation threshold (e.g. activation Tx), then in step 316 the AS node 300 lowers the activation threshold (e.g. activation Tx) and restarts the processing steps 311 and 312. As an option, a maximum number of cycles, and/or the activation threshold value, may be defined by the user or may be based on historical values.

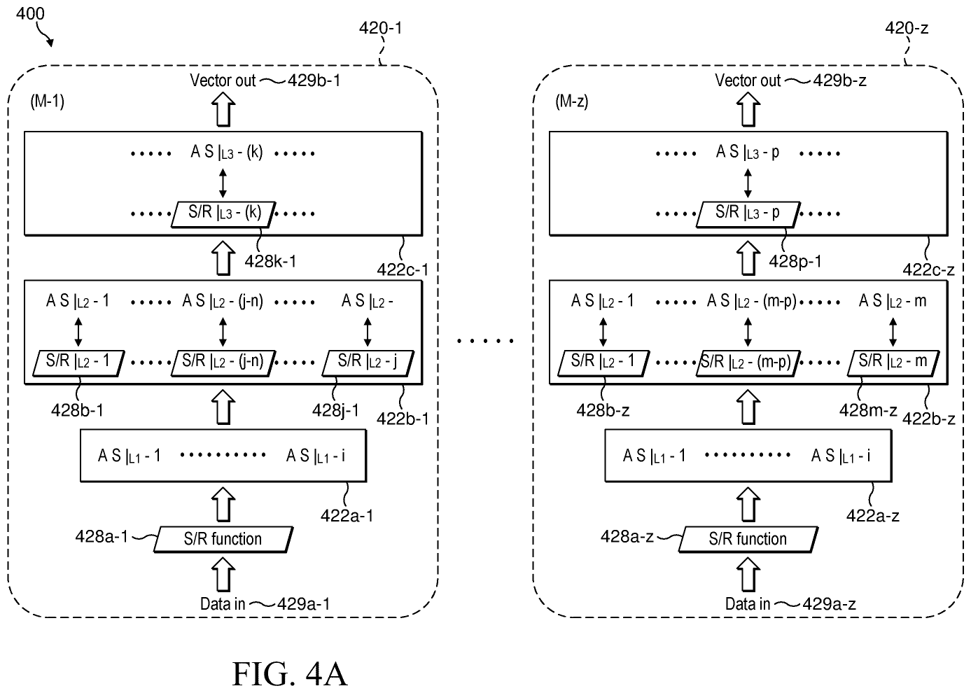

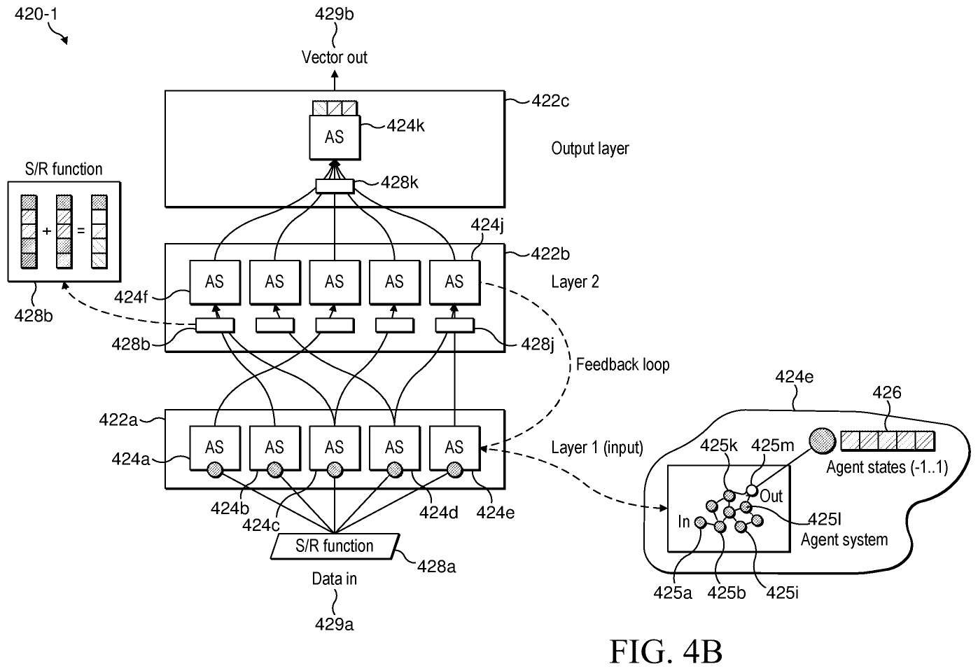

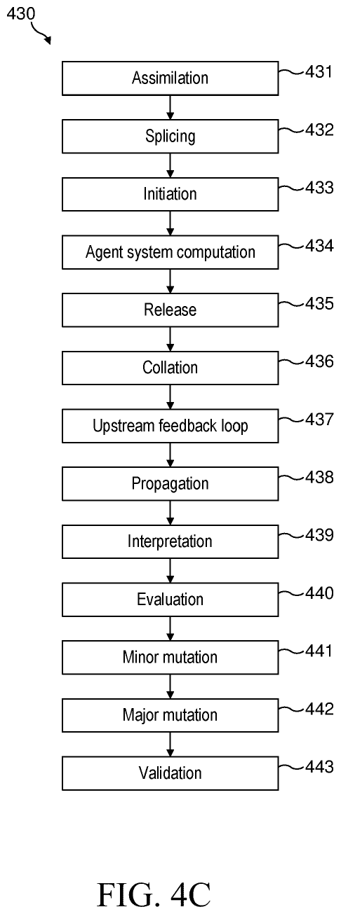

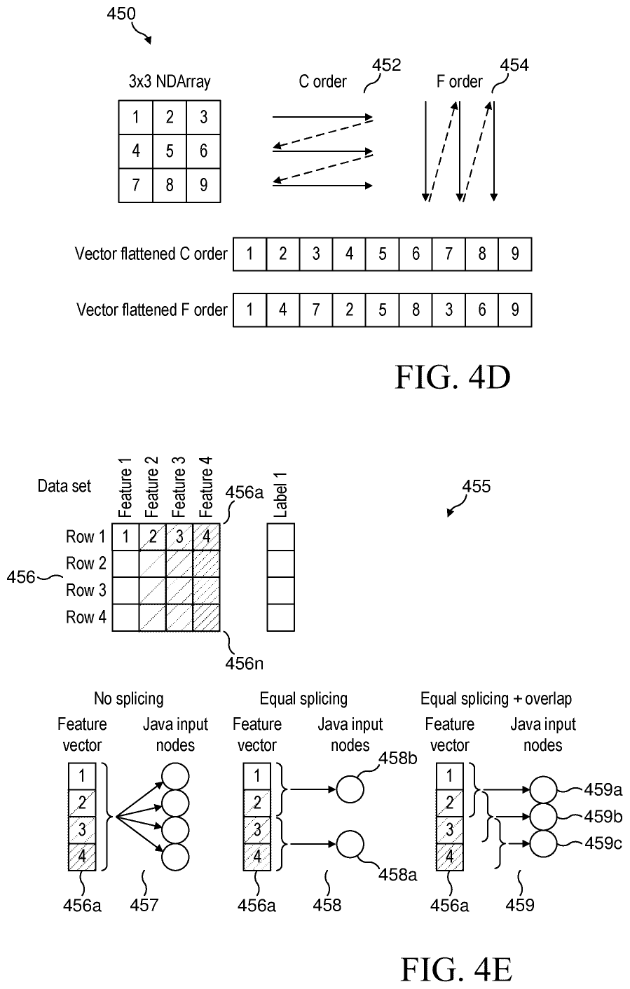

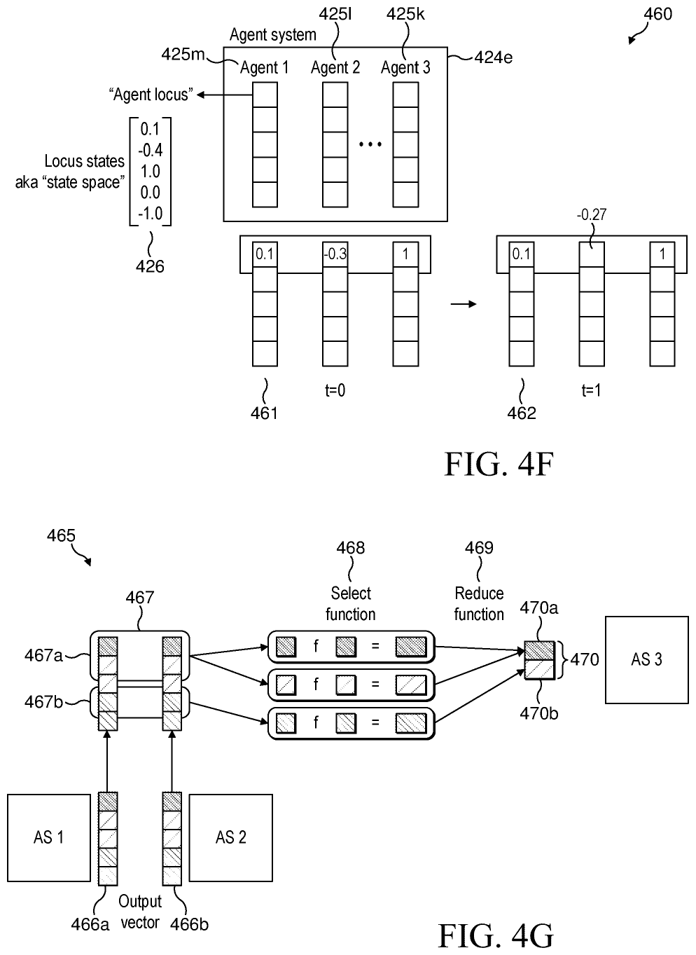

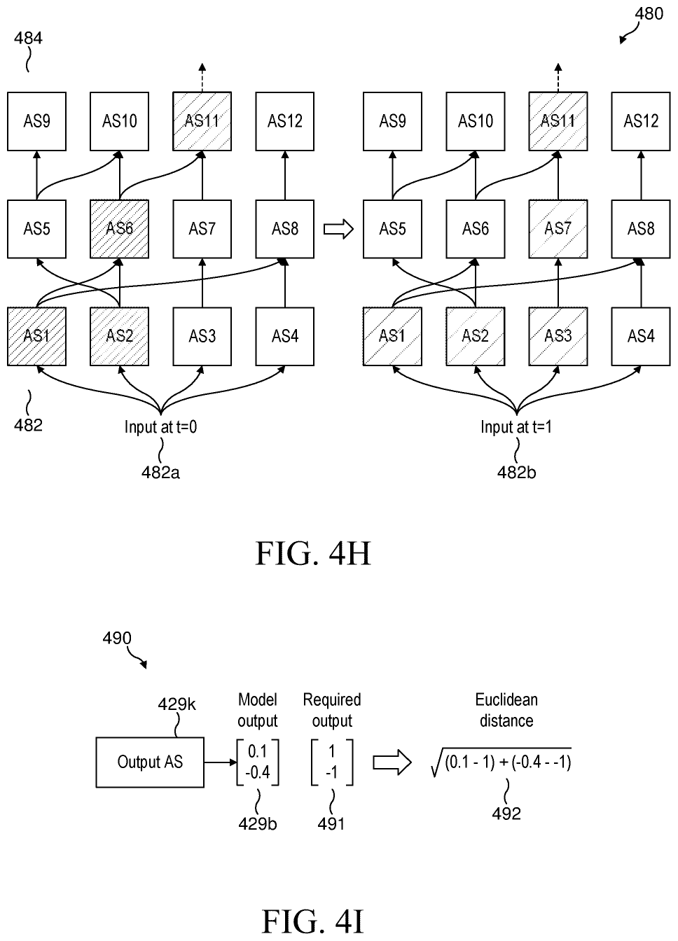

FIGS. 2a to 3b describe some examples of how to train an AM 200 and corresponding ASs for input to the fusion and/or model integration system 100 as described with reference to FIGS. 1a and 1b. FIGS. 4a to 6c are schematic and flow diagrams illustrating a specific example of training one or more AMs that are each configured for modelling an aspect of a complex system, where the aspects may be different aspects of the complex system, and integrating said one or more AMs into a fusion model or IM 109 for more accurately modelling the complex system based on said aspects. Although the model fusion/integration system, AMs and/or resulting fusion model are described with reference to FIGS. 1a to 3b for use in modelling complex systems and/or solving complex problems in various fields or domains including the medical or clinical fields/domains, this is by way of example only and the invention is not so limited, it is to be appreciated by the person skilled in the art that the model fusion/integration system, AMs and/or the resulting fusion model may be configured and hence applied to any other type of complex system and/or complex problem in any other field or domain such as, without limitation, for example, data informatics; biomedical and/or chem(o)informatics; oil/gas detection; geographical fields; space and/or satellite fields; agricultural and/or any other technical field in which a complex system may be modelled with multiple AMs that may be related and fused together using the model fusion/integration system to result in fusion model associated with the complex system and/or problem for, without limitation, for example analysing, detecting, predicting and/or classifying and the like.

FIG. 4A is a schematic diagram illustrating an example complex system 400 that is modelled by a plurality of AMs 420-1 to 420-z for fusing into a fusion model/integrated model according to the invention. The complex system 400 may include at least two agent model(s) 420-1 to 420-z for modelling the complex system. Each agent model 420-1a (AM) including a plurality of agent system (AS) node(s) (e.g. AS|_{L1}-1 to AS|_{L1}-i, AS|_{L2}-1 to AS|_{L2}-j, AS|_{L3}-k etc.). Each of the AS node(s) (e.g. AS|_{L1}-i) include a plurality of agent units (AUs) (not shown) and a set of AS rules governing the plurality of AUs. Each AU of the plurality of AUs is connected in an AU network to at least one other AU of the plurality of AUs. Each AM 420-1 of the AMs 420-1 to 420-z includes a corresponding input layer 422a-1 of the input layers 422a-1 to 422a-z, each of which includes a set of AS nodes (e.g. AS|_{L1}-1 to AS|_{L1}-i) of the plurality of AS node(s) (e.g. for AM 420-1 the input layer 422a-1 includes AS|_{L1}-1 to AS|_{L1}-i, AS|_{L2}-1 to AS|_{L2}-j, AS|_{L3}-k etc.). Each AM 420-1 of the AMs 420-1 to 420-z includes a corresponding output layer 422c-1 of the output layers 422c-1 to 422c-z, each of which includes at least one AS node (e.g. AS|_{L3}-k) of the plurality of AS node(s) (e.g. AS|_{L1}-1 to AS|_{L1}-i, AS|_{L2}-1 to AS|_{L2}-j, AS|_{L3}-k etc.). Each AM 420-1 of the AMs 420-1 to 420-z also includes one or more corresponding intermediate layers 422b-1 to 422b-z, each of which includes another set of AS node(s) (e.g. for AM 420-1 an intermediate layer 422b-1 includes AS|_{L2}-1 to AS|_{L2}-j) of the plurality of AS node(s)). It is noted that each of the AMs 420-1 to 420-z may have a different number of intermediate layers, and a different number of ASs in each layer, which may result from the types of training used to generate and/or create each of the AMs 420-1 to 420-z. Each of the AMs 420-1 to 420-z is trained to model one or more portion(s) of the complex system 400 using a corresponding labelled training dataset. Each of the AMs 420-1 to 420-z may be trained to model a different portion of the one or more portion(s)/aspect(s) of the complex system 400 using a different corresponding labelled training data set.

Each AM 420-1 is adapted, during training, to form: an agent rule base comprising one or more sets of AS rules; and an agent network state comprising data representative of the interconnections between the AS nodes (e.g. AS|_{L1}-1 to AS|_{L1}-i, AS|_{L2}-1 to AS|_{L2}-j, AS|_{L3}-k etc.) of the input, output and intermediate layer(s) 422a-1 to 422c-1. The agent rule base and agent network state are generated during training and configured for modelling said portion(s) of the complex system 400.

As described above with reference to FIGS. 1a to 3b, although the complex system 400 includes a plurality of AMs 420-1 to 420-z for modelling the various aspect(s) of the complex system, the AMs 420-1 to 420-z may be further optimised by performing a fusion or integration of the AMs 420-1 to 420-z to form a jointly optimised fusion model/integration model that more accurately and succinctly models the complex system 400. Briefly, the AMs 420-1 to 420-z may be input to a fusion/integration model process (not shown) that is configured to: determine an intersecting rule set between the agent rule bases of at least a first trained agent model 420-1 and a second trained agent model 420-z. The AS node structures from each of the different layers 422a-1 to 422c-z of the AMs 420-1 to 420-z and interconnections therebetween that are associated with the intersecting rule sets between the agent rule bases of each AS node structure are merged between the at least first and second trained agent models 420-1 and 420-z to form a first integrated/fusion AM based on combining those one or more layer(s), AS node(s), and/or AU(s) of the first and second trained AMs 420-1 and 420-z that correspond to the intersecting rule set. Should there be further AMs 420-1 to 420-z that are used to model the complex system 400, any intersecting rulesets/agent rule bases of the remaining AMs 420-1 to 420-z and/or the current merged first and second AMs 420-1 and 420-z and/or the first fusion/integrated model, may be determined which may be merged in a similar fashion, where the AS nodes, AUs, and/or layers of each AM with intersecting rule-bases/rulesets may be merged. This may be iterated over all of the AMs 420-1 to 420-z to form a resulting integrated/fused AM. The resulting integrated/fused AM may be further updated based on one or more validation and/or training labelled datasets associated with each of the plurality of AMs 420-1 to 420-z (e.g. at least the first and second trained AM(s) 420-1 and 420-z) until the resulting fused/integrated model is validly trained to form a trained fusion model (or integration model) that models the complex system 400. Thus, data representative of one or more data sources associated with each AM may be input to the fusion AM for modelling the complex system, which is processed by the fusion AM, and which outputs data representative of modelling the complex system, e.g. an indication, prediction or classification based on the input one or more data sources.

For example, the complex system 400 to be modelled may be detecting a tumour, disease or state of a subject from one or more images of the subject. The complex system 400 may be initially modelled by a plurality of detection AMs 420-1 to 420-z, each for detecting the disease or state of the subject based on different types of training datasets. A fusion AM may be configured for modelling the detection of the disease or state of the subject from one or more images of the subject, the fusion agent model derived from at least two of the AMs, each AM trained to model the detection of the tumour, disease or state of a subject from a different imaging source. As an example, the disease may be prostate cancer of a subject such that detection of prostate cancer of a subject is modelled by a plurality of prostate cancer detection AMs 420-1 to 420-z. Each prostate cancer detection AM is trained using a labelled training dataset including a plurality of labelled training data images of subjects in relation to detecting or recognising prostate cancer tumours from said labelled training data images. Each prostate cancer detection AM uses a labelled training dataset based on images output from the same type of imaging system that is different to the imaging systems used in each of the other prostate cancer detection agent model of the plurality of prostate cancer agent models. For example, each imaging system is a particular magnetic resonance imaging (MRI) system by a particular manufacturer. Thus, the resulting disease detection or subject state detection fusion AM may be input one or more images of the subject to the fusion AM, which processes the one or more images for detecting the tumour, disease or state of the subject based on the input one or more images of the subject, and outputs data representative of an indication, prediction, classification of whether the tumour, disease or state is present or detected from the one or more images of the subject, and/or an indication of the location and/or where in the image the disease and/or state of the subject is detected.

In another example, the complex system 400 may be based on detecting the state of the subject, such as bone fracture detection of the subject that is modelled by a plurality of bone fracture detection AMs. Each bone fracture detection AM may be trained using a labelled training dataset including a plurality of labelled training data images of subjects from a data source in relation to detecting or recognising bone fractures from said labelled training data images. Each bone fracture detection agent model uses a labelled training dataset based on images output from the same type of data source or imaging system. Each labelled training data item is annotated or labelled as to whether or not said subject of the plurality of subjects has a bone fracture, and/or if present, further labels indicating the location of the bone fracture in the image and the like. Each bone fracture detection agent model is trained in relation to images associated with different data sources or imaging systems. Additionally, each imaging system may be a particular X-ray scanning system by a particular manufacturer. Thus, a fusion model/integrated model that jointly optimises the bone fracture detection AMs may be obtained by merging the trained bone detection AMs based on intersecting rulesets/agent rule bases and the like. The fusion AM may then be used by inputting data representative of said one or more data sources (imaging systems) associated with the subject to the fusion agent model for detecting the disease or state of the subject, and the AM fusion model outputs data representative of an indication of whether the disease or state is detected from the input one or more images associated with the subject.

Although the above examples relate to image processing for detecting a tumour, disease and/or state of a subject, multiple AMs may be trained from different but related training datasets that are configured to model one or more different aspects of the complex system. For example, the complex system 400 to be modelled may be, without limitation, for example liver disease detection of a subject. The complex system 400 may then be modelled by a plurality of liver disease detection AMs, where each liver disease detection agent model is trained using a labelled training dataset derived from a different data source associated with a plurality of subjects. Each labelled training dataset comprising a plurality of labelled training data items based on the different data source and annotated in relation to whether or not said plurality of subjects have liver disease. Each trained liver disease detection AM associated with a different, but related, aspect of the complex system of liver disease detection.

In particular, the plurality of liver disease detection AMs may include at least the liver disease detection agent models from the group of: a first liver disease detection AM trained based on a labelled training dataset including a plurality of labelled training data items associated with a plurality of subjects, each training data item corresponding to data representative of lifestyle and/or ethnic background data/fields of a subject of the plurality of subjects and the lifestyle and/or ethnic background data/fields annotated or labelled as to whether or not said subject of the plurality of subjects has liver disease; a second liver disease detection AM trained based on a labelled training dataset including a plurality of labelled training data items associated with a plurality of subjects, each training data item corresponding to data representative of the genetics of a subject of the plurality of subjects and annotated or labelled as to whether or not said subject of the plurality of subjects has liver disease; a third liver disease detection AM trained based on a labelled training dataset including plurality of labelled training data items associated with a plurality of subjects, each training data item corresponding to data representative of one or more proteomic blood markers of a subject of the plurality of subjects and annotated or labelled as to whether or not said subject of the plurality of subjects has liver disease; a fourth liver disease detection AM trained based on a labelled training dataset including a plurality of labelled training data items associated with a plurality of subjects, each training data item corresponding to data representative of medical history data/fields of a subject of the plurality of subjects and the relevant medical history data/fields annotated or labelled as to whether or not said subject of the plurality of subjects has liver disease; a fifth liver disease detection AM trained based on a labelled training dataset including a plurality of labelled training data items associated with a plurality of subjects, each training data item corresponding to data representative of a sonograph and/or imaging of the liver of a subject of the plurality of subjects and each sonograph and/or imaging annotated or labelled as to whether or not said subject of the plurality of subjects has liver disease and/or, if present, further annotated with labels indicating the location of portions each sonograph and/or imaging of where the liver disease/tumours/cells etc.; and/or one or more other liver disease detection AMs, each trained based on a labelled training dataset including a plurality of labelled training data items associated with a plurality of subjects, each training data item corresponding to data representative of modelling another aspect of the complex system for diagnosing liver disease and annotated or labelled as to whether or not said subject of the plurality of subjects has liver disease.

Thus, the first, second, third, fourth, firth liver detection AMs may be combined to obtain a fusion/integrated AM that models the complex system of detecting liver disease in a subject based on different but related data sources/data sets. The fusion/integrated AM may be obtained based on: determining each of the intersecting rule set between each of the plurality of agent rule bases of the liver detection AMs; merging the liver detection AMs (those that have intersecting rule sets/bases) to form a fused agent model based on combining those one or more layer(s), AS node(s), and/or AU(s) of at least two AM with corresponding intersecting rule set(s) and the like; and updating the fused AM based on one or more validation and training labelled datasets associated with each of the at least two AM(s) until the fused AM is validly trained, where the trained fused agent model is the fusion agent model.

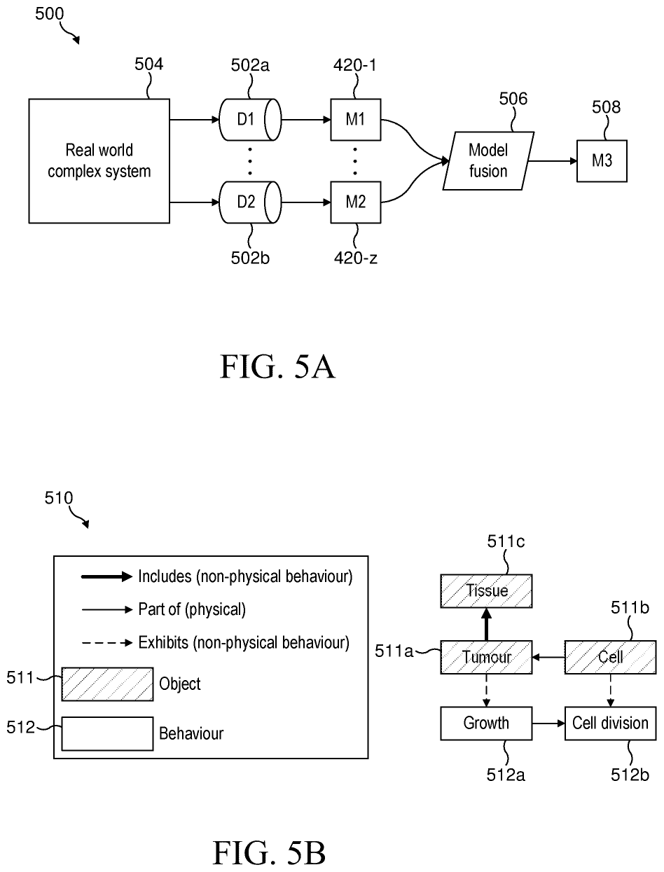

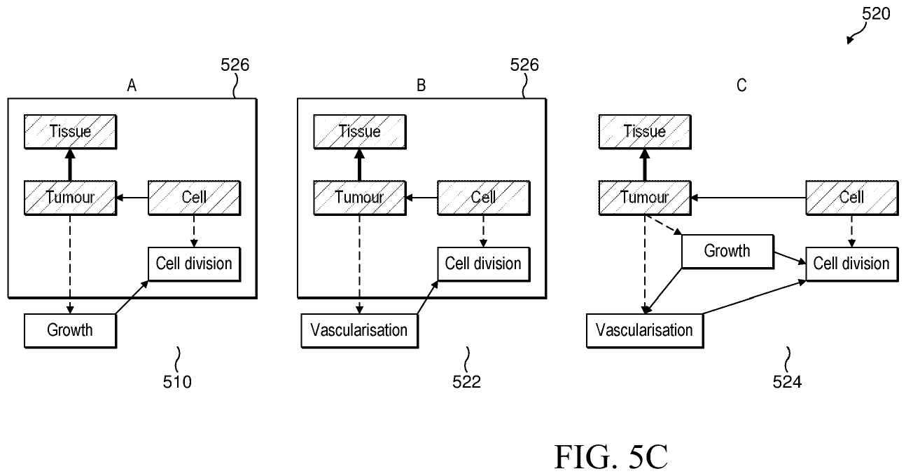

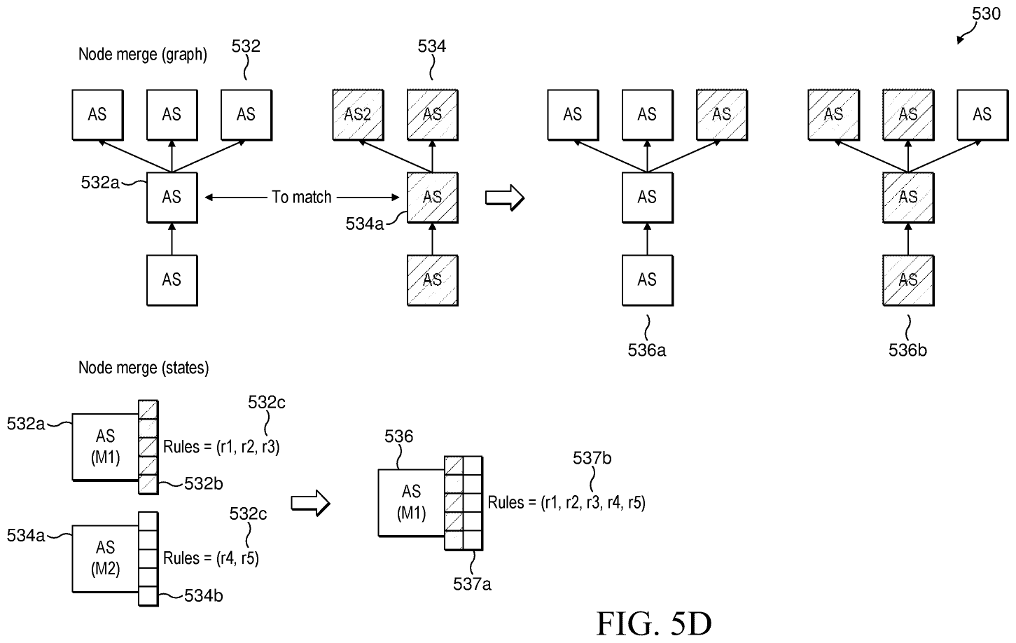

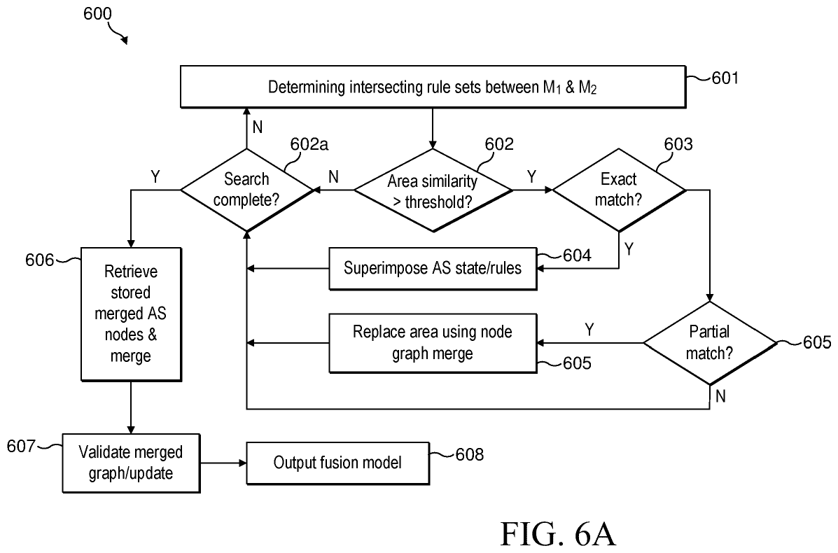

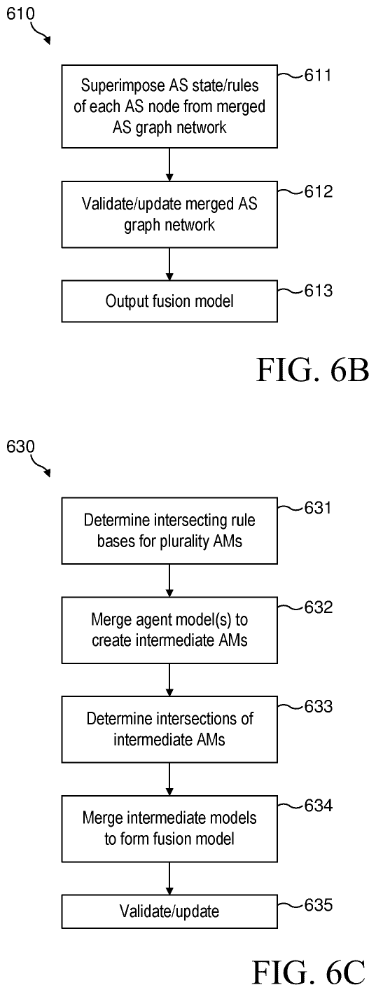

The resulting fusion AM that models the complex system 400 is obtained from determining intersecting rule-sets/rule bases of each of the AMs 420-1 to 420z that model an aspect, or related aspect of the complex system 400, where the related AS node structures, AU structures, connections between layers 422a-1 to 422c-1 and the like are merged and retrained to form the resulting fusion AM. FIGS. 4c to 6c outline a specific implementation of AM model and also the fusion (or model integration) process to achieve a jointly optimised fusion model for modelling a complex system 400 according to the invention. FIGS. 4a to 4j are used to describe the structure and training of an AM model and FIGS. 5a to 6c describe the process of obtaining a fusion AM 400 from a plurality of AM models 420-1 to 420-z for more accurately modelling a complex system 400 and the like.

FIG. 4B is a schematic diagram illustrating an example structure of AM 420-1 of FIG. 4A for use in the model fusion/integration process according to some embodiments of the invention. Each of the AMs 420-1 to 420-z may be based on the AM 420-1 of FIG. 4A but some of which may have the same and/or a different number of intermediate layers, the same and/or a different number of ASs, the same and/or a different number of AUs and the like. For simplicity, the “−1” label has been removed from the references of each of the features and/or components when illustrating AM 420-1 of FIG. 4B. The features and/or components of the AM model 420-1 as described with reference to FIGS. 4a to 4j may be used to further modify the features and/or components of the AM model 200 and/or process 210 as described with reference to FIGS. 2a to 2c, combinations thereof, modifications thereto and/or as herein described. For simplicity, the AM model 200 of FIG. 2A is reproduced for convenience.

Referring to FIG. 4B, as described previously with respect to FIG. 4A and/or FIG. 1A to 3B, the AM 420-1 is typically trained to model an aspect of a complex system (e.g., the detection of the disease or state of a subject from a data source including data of the subject). This is achieved by the AM 420-1 being structured to include a plurality of layers 422a-422c, each of the layers including a set of Agent System (AS) node(s) from plurality of AS node(s) 424a-424k. Each of the AS node(s) 424e includes a plurality of agent units (AUs) 425a, . . . , 425i, . . . , 425m and a set of AS rules or states 426 governing the plurality of AUs 425a-425m. Each AU 452a of the plurality of AUs connected to at least one other AU 425b of the plurality of AUs 425a-425m to form a network or graph of AUs 425a-425m as illustrated in FIG. 4b. The AM 420-1 includes an input layer 422a including a set of AS nodes 424a-424e of the plurality of AS node(s) 424a-424k, an output layer 422c including at least one AS node 424k of the plurality of AS node(s) 424a-424k, and one or more intermediate layer(s) 422b, where each of the intermediate layer(s) 422b each includes another set of AS node(s) 424f-424j of the plurality of AS node(s) 424a-424k. The AM 420-1 includes a plurality of Select/Reduce (S/R) functions 428a-428k that each is coupled to an input of one of the AS nodes 424a-424k. Each S/R function 428a-428k may operate to ensure the size of the output vectors from previous AS nodes 424a-424k is compatible with the input vector size required by the corresponding AS nodes 424a-424k coupled thereto. The AM 420-1 is trained to model an aspect of a complex system (e.g. the detection of the disease or state of a subject) based on a corresponding labelled and/or annotated training datasets, which include labelled training data items from a data source. The agent model 420-1 is adapted, during training, to form an agent rule base that includes one or more sets of agent system rules and an agent network state, which includes data representative of the interconnections between the AS nodes 424a-424k of the input, output and intermediate layer(s) 422a-422c, where the agent rule base and agent network state are generated during training of the AM 420-1 and configured for modelling said portion(s) of the complex system 400.

In FIG. 4B, the AM 420-1 is illustrated as having a single inner layer/intermediate layer 422b. It is to be appreciated that the AM 420-1 may have multiple inner/intermediate layers 422b that are interconnected with the other one or more inner layers (not shown) and/or the output layer 422c. FIG. 4B shows how an initial model topology looks, which is structured in a similar manner as an artificial neural network (ANN), with input and output layers 422a and 422c that sandwich an internal arbitrarily large layer set 422b. The structure illustrated in FIG. 4B is typically used to illustrate and describe the structure of the AM 420-1, but a mature or trained AM 420-1 (also called a mature kernel) has a different structure and may look more convoluted and/or irregular due to the changes made to the structure of the AM 420-1 during the training process. For example, as illustrated in FIG. 4B, every AS 424a-424k may be, without limitation, for example connected to every other AS in the layers above that AS and/or interconnected to every other AS in the layers below it, which may be an initial state prior to training, but as the AM 420-1 evolves, during training, the interconnections between AS nodes 424a-424k can be broken and remade.

FIG. 4C is a flow diagram illustrating an example AM training process 430 for use in training an AM 420-1 or each of AMs 420-1 to 420-z as described with reference to FIGS. 4a to 4b according to the invention. FIG. 4C may further modify the features and/or steps of process 210 of FIG. 2B for use in training AMs 420-1 to 420-z according to the invention. For simplicity, reference numerals of FIGS. 4a and 4b may be reused for similar or the same components, features, steps and the like. As well, FIGS. 4a and 4b will be referred to for simplicity. In the following, the flow of data (e.g. vectors) along the interconnections from the input layer 422a, through intermediate layer(s) 422b up through to the output layer/node 422b/424k is termed to be data flowing in the downstream direction. Conversely data flowing from the output layer/node 422b/424k down through the intermediate layer(s) 422b to input layer 422a is termed upstream. The AM training process 430 of the AM 420-1 may include one or more of the following steps of:

In step 431, data is assimilated from a data source and vectorised for data input as an input data vector 429a.

In step 432, the input data vector 429a is spliced in a configurable manner for input to the AM 420-1;

In step 433, each splice is propagated to each of the AS nodes 424a-424e of an input layer 422a via a summarizing select/reduce (S/R) function 428a;

In step 434, each of AS nodes 424a-424k performs agent system computations for transforming the input vector into another vector (possibly of a different size) and depending on the agent system's firing threshold, either the vector may be left as is or is modulated to “weaken” the output;

In step 435, the output agent vector(s) may be released from each AS node 424a-424e of the input layer 422a, where the new vector propagates downstream to all connected agent system nodes 424f-424j of the next layer, e.g. the intermediate layer 422b;

In step 436, before each of the receiving AS nodes 424f-424j processes the new vectors generated from the previous layer (e.g. input layer 422a), each receiving AS node 424f-424j must wait for all incoming connections from other upstream AS node(s) 424a-424e of a previous layer (e.g. input layer 422a) to also send their output vectors, thus the collation of input vectors for each AS node 424f-424j takes place, which is performed by the S/R function 428b-428j connected to the input of each AS node 424f-424j, respectively. Once all vectors from the previous layer are received at each AS node 424f-424j, said each AS node can process the input vectors and “fire” (i.e. perform the processing in step 434). It is noted that collation requires the S/R computation to be performed, e.g. S/R functions 428b-428j;

In step 437, an upstream feedback loop is also performed, where when an AS node 424f fires (e.g. outputs a vector to the next layer 422c), this AS node 424f will also send its output vector message to a connected upstream AS node 424b and/or 424c of the input layer 422a and these AS nodes 424b and 424c that receive this output vector message are configured to lower their firing thresholds. This is performed for all other AS node(s) 424g-424j of the intermediate layer 422b in respect of the connected AS nodes 424a-424e upstream in the previous layer, e.g. input layer 422a. Furthermore, with more than one intermediate later, this will be performed for each AS node that “fires” in each intermediate layer in respect of the connected upstream AS nodes of a lower intermediate layer above the said more than one intermediate layer that is above the input layer 422a;

In step 438, propagation is performed in which steps 424 to 437 are repeated throughout the AS node network and/or interconnected layers 422a-422b until the output AS node 424k of the output layer 422c receives and collates all the input vectors from one or more AS nodes 424f-424j of one or more intermediate layers 422c, where the output AS node 424k performs its own computations as in step 434. Thus, as the output AS node 424k does not receive strength signals from downstream node, because it is in the final AS node, so there is nothing downstream of it, the output AS node 424k does not have a firing threshold. Thus, it simply performs the AS computations based on the collated/Reduced input vectors via the S/R function 428k and passes the resulting vector output 429b to an output interpreter in step 439;

In step 439, the resulting output vector 429b is interpreted based on an output interpreter (not shown). Depending on its configuration, the output interpreter may modulate the output vector 429b before passing it on to an evaluator (not shown). This modulation/interpretation structure may be configured to assign the output vector 429b a label and/or annotation and the like such as the labels or annotations of the training data set being used to train the AM 420-1. The modulation/interpretation structure may be base one, without limitation, for example a simple computational structure such as, without limitation, for example converting [0.7,−0.9] to a YES/NO output based on a ±0.5 cut-off or it could be something more complex such as, without limitation, for example an ANN that interprets a large output vector into a classification and the like. The modulation/interpretation structure may thus output a predicted label in relation to the output vector 429b—the label may be within the label space of that of the training data set label space being used to train the AM 420-1;

In step 440, the modulated/interpreted output vector is evaluated, e.g. using an evaluation component, which may include, without limitation, for example a cost function (or evaluator) that calculates the distance or error between the predicted label associated with the output vector 429b and the actual label of the corresponding labelled training data item, which was vectorised as the input vector 429a, to produce, without limitation, for example data representative of an error value as a single number between 0 and 1;

In step 441, the AS node network that includes all the interconnected AS nodes 424a-424k and/or interconnected layers 422a-422c and/or AUs of each of the AS nodes 424a-424k may then be updated based on the error value of step 440. The update may take the form of a so-called “Minor Mutation”, where the error value is used to make modifications to the AS nodes and/or AUs of AS nodes of the AS network. This may simply be lowering/raising the firing thresholds of the AS nodes and/or AUs and the like.

In step 442, steps 432-430 are repeated for further labelled training data items in the labelled training data set or these steps are repeated for the same labelled training data item (e.g. the same data point) for a set number of cycles or iterations; This is performed until the labelled training data set has completed and/or until a minimum error rate has been achieved;

In step 442, should the minimum error rate not be achieved or a number of cycles is performed where the minimum error rate is not achieved, then a so-called large update or “Major Mutation” may be performed at certain configurable cycle point(s) (for example at an epoch completion), where more disruptive mutations of the AS network topology are performed/created before repeating steps 432 to 442 and/or before continuing onto step 443. The disruptive mutation may include, without limitation, for example randomly adjusting all the interconnections between AS nodes, AUs and/or layers. Thus, this may be performed should the previous AS network not be converging to a minimum error value.

In step 443, a validation is performed based on a validation data set, which may include repeating steps 431 to 440, but where steps 441 and 442 are not performed. The aforementioned steps are repeated with the validation data set (skipping steps 441 and 442) to evaluate the performance of the AM 420-1 in relation to out-of-sample data sets (e.g. data sets with known labels on which the AM 420-1 has not been trained).

In the example, each labelled training data item (e.g. data point) may run through the initial AM 420-1, at least once via steps 431-442. As a result, the AM 420-1 may be run for a set number of repetitions thereby allowing a certain error threshold and an error value to be reached, consistently. In a further example, the upstream feedback loop of step 437 may be switched off completely thereby allowing vector data to only propagate downstream with the only “feedback” being the propagation of the error value that elicits changes in the AS network topology and rules. In this further example, the upstream feedback loop may be modulated so that it propagates further than just a single upstream layer of nodes. Optionally it can also diminish as the signal travels further out. Additionally, the upstream feedback loop may be switched on and steps 432-439 may be repeated for the same training data item (e.g. data point) for a fixed number of cycles until a certain criteria is satisfied.

The flow of execution of AM training process 430 may be further modified based on the following configurations such as, without limitation, for example one or more from the group of: each data point (or labelled training data item) runs or is processed through the AS network of the AM 420-1 at least once; each data point (or labelled training data item) runs or is processed through the AS network of the AM 420-1 for at least a set number of repetitions; each data point (or labelled training data item) runs or is processed through the AS network of the AM 420-1 until a certain error threshold is reached over all data points and/or data items that in the labelled training dataset that is used for training AM 420-1; and/or each data point (or labelled training data item) runs or is processed through the AS network of the AM 420-1 for a consistency in error value is reached or plateau in the error curve is reached.

As an option, the system may be configured in which vectors are only fed downstream and the only “feedback” is the propagation of the error that elicits changes in AS network topology and rulesets/rule bases etc.; upstream feedback loop can be modulated so it propagates further than just a single upstream layer of nodes. Optionally it can also diminish as the signal travels further out; when upstream feedback loop is switched on, steps 432-439 are repeated for the same data point for a fixed number of cycles until certain criteria are satisfied.

Further additional modifications to the features and/or functions of each of steps 431 to 443 of AM training process 430 will be further described and/or elaborated on with reference to FIGS. 4d to 4j in the following description, where FIGS. 4a to 4c may be referred to in the following description.

The assimilation step 431 is configured to consume data from the data source (e.g. imaging scanners/medical imaging systems outputting image data) and prepares, processes, the data for the AM 420-1 and/or the plurality of AMs 420-1 to 420-z. In fact the assimilation process of the assimilation step 431 may be similar or the same in relation to other machine learning algorithms. This is also known as extraction, translation, load (ETL) which is the process by which data is consumed and made available. Essentially it is to make sure that the incoming data is formatted in the appropriate data structure for ingestion to the AM 420-1. For example, the assimilation process ensures each data item and/or the data from a data source is normalised (e.g. normalised values between 0 and 1 or −1 and 1). Typically, in the AM 420-1 all loci (or elements of the vectors associated with the data) may take a value between −1 and 1. As an example, in a medical data context, a comma separate value (CSV) or comma delimited value file that contains, without limitation, for example certain physical human features—height, weight, limb and torso dimensions—and a single label for gender may be assimilated into a set of vectors such that a single vector (containing a single data row) post-normalisation would look like, by way of example only, [0.3, 0.1, 0.7, 0.3, 1, 0], where the 1,0] at the end represents the label [Male, Female]. The preceding elements include data representative of the feature elements.

Although the process 430 is described with reference to AM 420-1 of FIGS. 4a and 4b, this is for simplicity only and by way of example and the invention is not so limited, it is to be appreciated by the skilled person that process 430 may be applied or used for training each of the AMs 420-1 to 420-z for modelling their corresponding aspect(s) of the complex system 400.

FIG. 4D is a schematic diagram illustrating an example of flattening process(es) 450 for use in the splicing step 432 of FIG. 4C. In the splicing step 432, which is post-ETL, the input vector 429a needs to be fed into the AS network topology via the S/R function 428a and input layer 422a. Thus, depending on the source data type, the input vector 429a may be arbitrarily nested. For example, in the above CSV example, a simple CSV file is a 1-dimensional array, but for images that have a Red/Green/Blue values for every pixel the 1-dimensional array vector may be nested based on, without limitation, for example: [[0.32,0.56,0.77], [0.11,0,1], [0.44,0.87,0.4] . . . ] Thus, the input data vector 429a may be a 1-dimensional vector, which may be fed one at a time into the input layer 422c. Given that most of the time the AM 420-1 may only deal with 1-dimensional vectors at a time, such nesting may need to be flattened first as shown in FIG. 4E. For example, two types of flattening may be performed C-order or F-order vector flattening 452 or 454. Once the input data vector is flattened the entire data row can be traversed and chopped (i.e. spliced) with a predetermined pattern to suit the current AS topology of the AM 420-1 as described with reference to FIG. 4E.

FIG. 4E is a schematic diagram illustrating various example splicing subprocess(es) 455 for use in the splicing step 432 of FIG. 4C. The AM 420-1 may use one or more configurations of splicing strategies for use on the input data set 456 (the rows represent a plurality of input data vectors 456a to 456n) such as without limitation, for example: No Splicing 457, where an input data vector 456a in its entirety is copied to every input AS node 424a-424e of the input layer 422a; Equal Splicing, where the input vector is divided equally (with leftover appended to the last splice) to every node, e.g. the vector {1, 2, 3, 4} is divided equally into subvectors {1, 2} and {3, 4} each of which are applied to a different input AS node 424a to 424e of the input layer 422a; and/or Equal Splicing+Overlap 459, where a sliding window may be applied over the input vector 429a and a fixed length copy 459a-459c is sent to every input AS node 424a-424e of the input layer 422a. Although three splicing strategies 457, 458 and 459 are described in FIG. 4E, this is by way of example only and the invention is not so limited, it is to be appreciated by the skilled person that other and/or more complex splicing strategies and/or derivations are possible depending on the type of data source and data sets/input data vectors that are produced, combinations thereof, modifications thereto and/or as the application demands. For example, for images the input vector 429a may be a 2-dimensional array, so to flatten and splice, there may be an x- and y-axis sliding window arrangement for enabling a 1-dimensional input vector representing the 2-dimensional array/matrix to be input to the AS nodes 424a-424e of the input layer 422a.

In the initiation step of step 433 of process 430, the state space (e.g. the network of AUs 425a-425m representing each AS) of each AS node in the input AS nodes 424a-424e is typically fixed during a training cycle (until it gets mutated at the mutation step). This size need not be the same size at the input vector splice. In fact this is true for all interlayer vector transport: the receiving AS state space will likely not be equal to the incoming vector size that is input to the AS node. The AM 420-1 takes care of this inequality with a plurality of Select/Reduce (S/R) functions 428a-428k that essentially exists between each connected AS node 424a-424k. For example, each S/R function 428b is coupled to the input of an AS node 424f as illustrated in FIG. 4B. As a special case, the S/R function 428a acts as an input to each of the AS nodes 424a-424e of the input layer 422a, where this S/R function 428a may be adapted to the splicing strategy of step 432 for distributing portions of the input vector 429a to each input AS node 424a-424e accordingly.

For example, for the input AS nodes 424a-424e of the input layer 422a, the S/R function 428a may be specialised/modified to perform the following reduction on the incoming input vector 429a vᵢ: a) If input node state size=1, then v→[vᵢ]; b) If input node state size=2, then v→[vₘᵢₙ, vₘₐₓ]; c) If input node state size=3, then v→[vᵢ, vₘᵢₙ, vₘₐₓ], and d) If input node state size >=4, then v→[vᵢ, vₘᵢₙ, vₘₐₓ, d(v)], where d(v) is a distribution summary function that tries to describe the distribution of the input vector 429a as if it were a data series, and outputs a vector of the desired size.

There may be many derivations of d(v), however currently it is restricted to a bucket ranking so that given state size s, that resulting vector will be of length sᵥ=s−3, where elements of sᵥ contain the proportion of distribution (between 0 and 1). For example, this may be represented more formally by the following process:
1. Given v of length n where n>3
2. A new vector is required to occupy in the input vector, sᵥ=s−3
3. Assume all incoming values, vᵢ, are between −1 and 1 (and if not, they should be normalised)
4. Calculate average, minimum and maximum of vᵢ (vᵢ, vₘᵢₙ, vₘₐₓ) (which will occupy the first 3 loci of the result vector)
5. Let boundary

$$b = \frac{1}{{s}_{v}}$$

6. For each value vᵢⱼ in vᵢ:
   a. Calculate

$${v}_{ij 1} = \frac{{v}_{ij} + 1}{2}$$

b. Calculate

$${v}_{ij 2} = r ( \frac{{v}_{ij 1}}{b} )$$

(where r is the Round Down function)
c. Calculate which “bucket” vᵢⱼ₂ belongs to in sᵥ
7. For each sᵥ, calculate the proportion of hits in each and update into sᵥ.

In the agent system computation step of step 434 of process 430, the agent system node or AS node is one of the fundamental components of the AM 420-1 because it “houses” some of the self-emergent intelligence of the AM 420-1 system as well as the rule base that will be used later for fusion and/or integrative modelling to generate a fusion or integrated model from multiple AMs 420-1 to 420-z. As already described with reference to FIGS. 1a to 4e, the AS node includes a plurality of agent units that are interconnected together. An AS node forms a multi-agent system (MAS) and further has the following components and/or properties, with reference to AS node 424e of FIG. 4B, such as, without limitation, for example: an AS node 424e is a MAS that is composed of n agent units 425a-425m that are interconnected together to form an AS node 424e; each AS node 424e has a designated input agent unit 425a, A₀, that receives messages (input vectors) from upstream (upstream could be other AS or the Initiation S/R); each AS node 424e has a designated output agent unit 425m, Aₙ₋₁, that propagates messages (vectors) downstream (downstream could be other AS 424j of another layer 422b or the final output S/R 428k of the output AS node 424k); each agent unit 425m is primed with some interaction rules, Rₙ, and every agent unit 425a-425m within a single AS node 424e is primed with the same rule base; each agent unit is primed with a vector of states 426, where the vector of states has a fixed length during a single learning cycle; all agent units 425a-425m are configured to have, without limitation for example identical length state vectors 426; in the learning phase the state space 426 of the vectors of an agent unit is either randomly allocated or allocated by some other heuristic; The AS node 424a has an activation threshold value, 0≤a_{τ}≤1, a current activation value 0≤a≤1, an activation function that modifies a, e.g. on a sigmoid curve (configurable), an activation threshold function that modifies a_{τ}, e.g. on a sigmoid curve (configurable), and a maximum number of cycles, c. It is to be appreciated that the structure of the other AS nodes 424a-424k of AM 420-1 may be based on, but are not necessarily identical to, the structure of AS node 424e.

That is, each agent unit in an AS node behaves independently of the whole, where the behaviour (state) of an agent unit at any time step is a deterministic function of its previous states (memory) and the state of the agent units in its adjacency (to a degree of freedom of 1 though it may expand in later iterations. This is called the neighbourhood function, N_{A}.

FIG. 4F is a schematic diagram illustrating an example agent node system 460 for use with the AM 420-1 (or each of AMs 420-1 to 420-z) of FIGS. 4A to 4E according to the invention. For simplicity, agent node system 460 is based on AS node 424e of FIG. 4B in which the agent units 425a-425m within the AS node 424e are governed by a set of rules, which are identical across all agent units 425a-425m. A simple example of a single rule for an agent unit 425m might be, without limitation, for example, take all the state locus 1 values of my neighbour agent units 4251 and 425k, calculate the average, and set that as my new state locus 1 value of the agent unit state vector 426. Each of the agent units 425a-425m may perform a similar rule to adjust their state vectors.

In essence, FIG. 4F describes an agent node system 460 with agent system node 424e made up of multiple agents 425a-425m where each agent of the multiple agents 425a-425m are “connected” (i.e. neighbours) to one or more other agent units of the multiple agents 425a to 425m. In this example, Agent unit 2 4251 only has two neighbours, Agent unit 1 425m and Agent unit 3 425k. Each agent unit has a state space vector composed of loci (e.g. vector elements). Each locus is a value in the range −1<=0<=1. Each rule operates on defined loci. So, at t=0, 461, an Average Rule operating on Agent 2 takes the neighbouring and self values (0.1, −0.3, 1), calculates the average and replaces that new value (0.27) into that locus for t=1, 462. Every rule is executed before this agent node system 460 can be timestamped as t=1.

There are a few rules and caveats for valid set of AS agent rules such as, without limitation, for example: a) A rule takes as input the entire state space of neighbouring agent units; b) Neighbouring agent units are determined by a connectedness between agent units and a neighbourhood function determines connectedness. State cannot be inferred for non-neighbours during rule execution; c) A rule may tap into agent historical state memory; d) A rule must only use, as its input, present and past state memory, i.e. calculations for time step t can only use states from time steps <t; e) A rule executed for a particular agent can only affect that agent's state; f) A rule and the states that it affects are tied to the data set that is being learned and its corresponding semantic network, which is useful for model fusion and/or model integration as described with reference to FIGS. 1a to 3b and/or FIGS. 5a to 6c.

As an example, the rulebase (or rule set) for the agent units of an AS node may include, without limitation, for example: 1) a Minimum rule, which takes the minimum of all selected states; 2) a Maximum rule, which takes the maximum of all selected states; 3) an Average rule, which takes the average of all selected states, where variations of this rule may include, without limitation, for example: a) median, b) mode, and/or c) fixed number-weighted average; and/or any other type of statistical averaging function; 4) a Sum rule, which takes the sum of all selected states (limit to −1, 1); 5) a Sum toroidal rule, which is similar to the sum rule, except treats the (−1, 1) number line as toroidal; 5) a Flip rule, which flips the sign (−ve to +ve, +ve to −ve) of selected states/loci; and/or 6) an Abs rule, which takes the absolute (+ve) value of all selected states/loci; and/or any other rule that is defined accordingly.

In steps 441 and 442 of the AM training process 430, during the optimisation Minor and Major Mutation phases, the AM 420-1 may adjust the rules and/or perform other rules to perturb the state vectors of the agent units by, without limitation, for example conjugating rules, e.g. Sum+Flip and the like.

Given that the agent system node and the agent unit network/rules have been described, the agent system computation step 434 of process 430 further includes the agent system node execution algorithm, which may be performed for each of the AS nodes 424a-424k of the AM 420-1. For example, for an agent system node 424e of the plurality of AS nodes 424a-242k in which the agent system node 424e includes a plurality of agent units 425a-425m (e.g. A₀ to Aₙ₋₁) interconnected together, the agent system node execution algorithm may be based on the following steps of:
1. Let t be the current time step, starting at 0.
2. The input AS node 424e receives a single vector at time step 0, v_{i,t=0}, that matches the required length.
3. If a<aₜ, go to step 6. Else, while cₜ<c:
   a. Set A_{0,t=0} 425a state to v_{i,t=0}
   b. Find the neighbourhood agents with N_{A0→N}=N_{A}(A₀)—this can be treated as a matrix of states.
   c. For each state locus, given Rₙ, calculate the new state rule, and set result A_{0,t=1}
   d. For A₁ to Aₙ₋₁ 425b-425m repeat steps a-c.
4. Take state A_{n-1,t=c-1} and propagate downstream (e.g. output as output vector of AS node 424e).
5. Optionally reset states of all A 425a-425m to a historical state, e.g. to the original state they were at before step 2.
6. (If the agent didn't fire) Modify the activation threshold downward with function a_{τ,t=1}=max[a(a_{τ,t=0}),0].
   The steps outlined in the above Agent System node execution algorithm perform a computation that represents a nonlinear calculation. Similarly, in the training phase of the AM training process 430, the flow of execution can be optionally modified so that the condition cₜ<c is replaced by an output condition, i.e. until a predetermined output vector is achieved.

In the release step 435 of process 430, the output vector of each AS node is simply sent to all connected downstream AS nodes of subsequent layers 422a-422c. Optionally, the activation level is heightened according to any function that modifies the signal in the boundary 0≤a≤1. A single AS node that receives multiple messages from an upstream AS node (e.g. incoming vectors) needs to be able to reduce those messages down to a vector length that matches its own input vector length. This is done by an S/R function 428b-428k.

FIG. 4G is a schematic illustration of an example S/R function 465 for use in each of the S/R functions 428b-428k of AM 420-1 of FIG. 4B and/or in AM training process 430 of FIG. 4C and the like according to the invention. FIG. 4G is an example of the collation phase of collation step 436. The S/R function 465 may be modified by one or more of the features of the S/R function 230 or S/R functions 428b-428k as described with reference to FIG. 2c or 4b and/or the S/R function 230 or S/R functions 428b-428k may be modified based on one or more features and/or components of S/R function 465 of FIG. 4G. In any event, the S/R function 465 defines the following, without limitation, for example, for every input vector locus 466a, 466b (e.g. the output vectors of AS 1 and AS 2): a) A source to target mapping of output vectors 466a, 466b from AS 1 and AS 2 (e.g. AS node 1 and AS node 2 output vectors) to input vector 470, which is input to AS 3 (i.e. AS node 3 of another layer): the loci to select from incoming output vectors 466a and 466b (i.e. a list of indices); b) A “select” function 468 that maps across the incoming output vectors 466a, 466b at the same locus and reduces that collection of states to a single state; and c) A “reduce” function 469 that resolves multiple states writing to the same target state and thus a fixed input vector 470 is provided to the connected AS node 3 (e.g. AS 3).

For example, FIG. 4G is an example of the collation phase in relation to AS node AS 3, where the input vector length of AS 3 is of length 2. Its S/R function 465 is configured to define, without limitation, for example the following: 1) Source to target mapping of source output vectors 466a-466b to input vector 470, where Indices 1 and 2 (see square box 467a) of incoming output vectors 466a and 466b map to index 1 (e.g. 469a) of the AS 3's input vector 470, Index 4 (e.g. square box 467b) of incoming output vectors 466a and 466b map to index 2 (e.g. 469b) of the AS 3's input vector 470. In this example the same origin indices of AS1 and AS2 contribute, without limitation, for example to the first and second loci 470a and 470b of the input vector 470. This need not be the case; for example, the locus 470b could be at a different index in another upstream AS node; 2) the Select function 468, where for index 1 (e.g. 470a) of the target input vector 470, the select function executes on the two pairs of loci of Indices 1 and 2 (e.g. square box 467a) of the two output vectors 466a and 466b to produce a single number for each index; and for index 2 (e.g. 470b) of the target input vector 470, the select function executes on the pair of loci (e.g. 467b) of the two output vectors 466a and 466b to produce a single number for the pair of loci at index 4; and 3) a Reduce function 468 in which for index 1 (e.g. 470a) of the target input vector 470 two pairs of loci of Indices 1 and 2 produced a single number for each index, thus the reduce function combines the two numbers to form a single number for the locus of index 1 (e.g. 470a) of the target input vector 470, thus the result from the select function is reduced to a single number; and for index 2 (e.g. 470b) of the input target vector 470, a reduction function is not required because the select function 468 already reduced it to a single number. The constituent S/R functions 428b to 428k connected to each of the AS nodes 424f to 424k can be almost any logical/mathematical function, so long as the output respects the state boundary −1≤x≤1.

FIG. 4H is a schematic diagram illustrating an example AS network state 480 for illustrating the upstream feedback loop of step 437 of the AM training process 430 according to the invention. In FIG. 4H, the direction of information flow is from bottom (input) 482 to top (output) 484. When at t=0, the first input 482a goes into AS1 to AS4 but AS3, AS4 and all descendants from those AS nodes (e.g. AS7, AS8) fail to fire (represented by no hatching) because of, in this example, a lack of signal. However, AS1, AS2 and AS6 fires (represented by close hatching) and inputs to AS 11 in the same time step such that AS11 fires strongly, which, in the next time step at t=1, then sends a feedback signal upstream (e.g. back towards the bottom input 482) to AS6 and AS7 and their descendants AS1, AS2 and AS3. This feedback signal is configured to decrease the excitation threshold of these descendent AS nodes AS6, AS7, AS1, AS2, AS3, where that decrease makes it more likely that these nodes will fire (represented by the spaced-apart hatching) on the next input—i.e. AS1, AS2, AS3 (which did not fire in t=0), AS6 and AS7 (which also did not fire in t=0) now all fire. Thus, the upstream feedback loop is configured to assist in getting AS nodes that do not fire to fire when a descendent AS node above fires strongly.

It is noted that the feedback loop is an optional step of the AM 420-1 and/or AM training algorithm/process 430 and is designed for sequential machine learning, e.g. time series data analysis or natural language processing, where there is a requirement to retain a certain memory capacity whilst the machine ingests the next data point/labelled training data item (the next time series step, or next word in the sentence).

When an AS node triggers or fires and produces an output vector, that output vector is sent to downstream AS nodes of subsequent layers that connect to the AS node. The output vector of the AS node that triggered is additionally sent to upstream AS nodes that did not cause that AS node to fire in the first place (i.e. dormant AS nodes). Thus, when an upstream AS node receives that message of the output vector, it applies, without limitation, for example the following adaptation to the agent units or agent unit network of the AS node: Modify the excitation level threshold upwards with function a_{τ,t=1}=min [a(a_{τ,t=0}), where the function a can be any mathematical function, without limitation, for example a sigmoid, straight line, step function and the like.

The purpose of the upstream feedback loop is to drive the learning optimisation process of the AM training process 430 in such a way that certain sections of the AS network coordinate recognition of certain sets of co-related features and therefore cluster together in the AS network. When a single feature is recognised, it “eases” the recognition of co-related features to enhance the overall pattern recognition of the AM 420-1.

In the propagation/interpretation of steps 438/439 of process 430, with reference to FIGS. 4B and 4C, the output vector messages that are output from one or more of AS nodes 424a-424j propagate as inputs to downstream connected AS nodes of subsequent intermediate layer(s), some of which may subsequently “fire” and output further vector messages such that they propagate onwards downstream through the layers 422a-422c of the AS network 424a-424k and coalesce at the output AS node 424k, where a single output vector 429b is generated.

For AM 420-1, the output vector 429b is not the same as that output, without limitation, for example an ANNs vector output, because it need not be a vector that matches the label vector length. For example, for a binary recognition ANN where the outcome is “Yes” or “No” the expected output vectors are actually data representative of “Yes” or a “No”, for example: [1,0]=YES, and [0, −1]=NO, where the output vector length is 2, and each locus is dedicated to a single label, where in this example, locus 1 is dedicated to “Yes” and locus 2 is dedicated to “No”. Rather, the interpreter module of the AM 420-1 and the AM training process 430 is configured to allow for an additional recognition step after the output AS node 424k produces its output vector 429b. For example, taking the binary example of the ANN, the AM 420-1 is typically configured to have a relatively long output vector, e.g. the binary vector could be, for example, 10-loci long. An additional deterministic function is applied to translate that 10-length vector into a Yes or No. This deterministic function may be, without limitation, another AM 420-z, another machine learning algorithm, an ANN or other neural network structure (e.g. autoencoder), or a simple heuristic and the like. Thus, the AM 420-1 usually has an expanded vector length, where the expansion of the vector length beyond what is assumed to be required (e.g. ANN assumed that a vector of length 2 was required for a “Yes”, “No” output) is found to actually aid the subsequent AM model fusion/integration process in that it builds into the fusion/integrated model and/or the AM 420-1 a possibility that there are as yet unknown states that may need to be optimised.

In the evaluation step 440 of AM training process 430, an evaluation component may perform a fitness evaluation of the AM 420-1, which is a measure of how far the prediction (post interpretation) is from the expected value given the feature set and labels of the labelled training dataset. This fitness evaluation (or cost function) may be determined by one of the many known distance or similarity measures such as, without limitation, for example Euclidean distance, Squared Euclidean distance, Hamming distance, Chebyshev distance, Manhattan distance, Minkowski distance, and/or any other type of distance and/or objective score, based on either Euclidean or non-Euclidean space, that summarises the relative difference between two objects in a problem domain such as the interpreted output vector and the label of the corresponding labelled training data item associated with the output vector of the AM 420-1. Given that all values in all loci may be, without limitation, for example restricted to −1≤x≤1, the distance or similarity measure can be normalised into the same bounds. The resulting fitness evaluation value may be treated like an error value as shown in FIG. 4I. FIG. 4I is a schematic diagram that illustrates an example evaluation component 490 for evaluating the interpreted model output of an AM 420-1 during training and/or validation steps of AM training process 430 and the like. For example, at the end of the training phase of the AM 420-1, the output AS 424k outputs an output vector 429b, which may have been interpreted, where the output vector is compared in the evaluation step 440 to the labelled training data item 491. In this example, the vector [1,−1] is a label of the labelled training data item 491, which is what output is actually required from AM 420-1. The model AM 420-1 produces an output vector 429b, where the evaluation component or step 440 performs a fitness evaluation and makes an error calculation using, without limitation, for example Euclidean distance measure 492, which is one of the most popular measures. Although Euclidean distance is described herein, this is for simplicity and by way of example only and the invention is not so limited, it is to be appreciated by the skilled person that any other function may be used such as, without limitation, for example a similarity measure, distance measure, error function, and/or cost function, combinations thereof, modifications thereto and/or as herein described and/or as the application demands. The fitness evaluation or the error calculation/cost function calculation may be used for adapting the AS nodes, AUs of each AS node, connections between AS nodes and/or layers and the like.

In steps 441 and 442, perturbations and/or adjustments to the AM 420-1 are made based on the output of the evaluation step 440 and the number of cycles and the like. These may be so-called minor mutations as described in mutation step of step 441 or major mutations in step 442. The minor and major mutations of steps 441 and 442 may be triggered by, without limitation, for example three configurable states that are set at the start of the execution of the AM training process/algorithm 430 and which may include, without limitation, for example: 1) After a set number of evaluations; 2) After the trajectory of the error curve plateaus or spikes (or some other configurable trigger); and/or 3) A combination of 1) and/or 2) above. There are various minor and major mutations that the AM training process 430 may make depending on the severity of the error value output from the evaluation process 440. Thus, based on the error value, a selection of candidate mutations are selected and then further filtered for suitability, where suitability is determined by a set of conditionals that are evaluated based on, without limitation, for example: a) Will the mutation permanently disrupt the flow of messaging up and down the AS network of the AM 420-1? b) Does the mutation violate S/R requirements that are already in place? and/or c) as an option, simply supply a stochastic value to determine whether or not to proceed.

For minor mutations in step 441 of process 430, the vast majority of mutations will be a simple rule modification that models locus-specific signals in the AUs of the plurality of AS nodes 424a-424k, which simple rule is called self adjustment. During the execution of a vector output messages flowing up the AS network of the plurality of interconnected AS nodes (i.e. downstream) to the output AS node 424k, a monitor may be configured to record the deltas of the signal at each vector locus. It is therefore able to determine which AS nodes contribute most/least to each output locus. Based on this information, a self adjustment rule is modified to inflate or deflate that particular locus value. The specific amount is determined by a contribution heuristic. For example, a contribution heuristic may be based on, without limitation, for example the following algorithm:
1. Determine the total number of AS nodes involved in contribution of the locus (t).
2. Determine the error of the particular locus (e).
3. Calculate e_{c}=m(e/t) for each AS node in a single random path from output AS node to input AS node, where m is a diminishing value as the degree of freedom from output AS node becomes greater.
4. Order e_{c} in descending order, and apply that value negatively or positively (as required) to that locus in the form of Self Adjustment rule.
   a. The Self Adjustment rule always executes after other AS rules have executed.
   b. This rule simply nudges the locus value towards the desired output value.
5. Only do step 4 for a configurable number of AS nodes.

In step 434 of the agent system computation of process 430, a modified execution flow was described for an AS node. Another way of achieving AS node optimisation is to use e_{c} to predetermine a desired output vector and let the AS node self-organise to produce that outcome. The steps for such an execution are based on, without limitation, for example the following:
1. Run step 3 of the agent system execution algorithm for 1 cycle.
2. At the end of the cycle:
   a. Calculate the distance of each locus from the desired locus value.
   b. Reorient the agent unit connections of the loci that are over-represented, move further apart (i.e. disconnect) vice versa for loci that are lightly represented (i.e. create connections).
      i. If the desired outcome is achieved within a given error bound, break out of loop.
   c. Run step 1.
      In this modified flow, activation thresholds are ignored.

In the major mutation step 442 of process 430, the mutations may be based on, without limitation, for example one or more of the following, where each of which can be modulated based on the severity of the error value: a) AS node internal reconnectivity, where agent units of an AS node are reconnected, including neighbourhood function mutations; b) S/R function mutations, which include, without limitation, for example swapping out functions of the S/R functions for other functions, modulating the parameterisation of those functions and the like; c) agent unit rule mutations based on, without limitation, for example modulating parameterisation of those functions/rules, deleting, creating, duplicating rules and the like, shuffling rule order, combinations thereof, modifications thereto, and/or any other rule mutation and the like as the application demands; d) agent unit state mutations such as, without limitation, for example randomising the agent unit state(s) and the like; e) agent unit mutations such as, without limitation, for example, modulating the cycle number, rotating input/output AS nodes, activation threshold modulations and the like or as the application demands; f) whole AS network reconnectivity such as, without limitation, for example breaking/creating connections, randomly breaking/creating connections between AS nodes and/or between AUs of an AS node and the like. Major mutations are typically large scale perturbations performed on the rules, rule state, agent state and/or connections between layers, AS nodes and/or AUs of AS nodes and the like.

In the validation step of step 443 of process 430, the mutation phases of steps 441 and 442 represents the “learning” phase of the AM training process 430 and/or algorithm governing training of AM 420-1. In the validation phase, the resulting kernel or AM 420-1 is tested against a known labelled data set that it has not yet experienced. In the validation phase the mutation steps 441 and/or 442 may be switched off.

Thus, at the end of the AM training process 430 as described with reference to FIGS. 4a to 4i, one or more AMs 420-1 to 420-z may be trained using different but related labelled training data sets to generate AMs 420-1 to 420-z that may each model a different aspect of a complex system 400 such that the output vectors of each of the AMs 420-1 to 420-z may be combined to provide an output observation associated with the modelling the output of the complex system 400 and the like. However, when only combining output observations together from multiple AMs 420-1 to 420-z and/or an ensemble of AMs 420-1 to 420-z to form an observable output in relation to modelling the complex system 400, such ensemble AMs 420-1 to 420-z result in suboptimal and/or biased output results because such combination of outputs do not take into account the possible relationships that may occur between the labelled training datasets of each of the AMs 420-1 to 420-z and/or possible relationships between structural components of each of the trained AMs 420-1 to 420-z and the like. This may be addressed by performing model integration/fusion as described with reference to FIGS. 1a to 3b on AMs 420-1 to 420-z that model a complex system. FIGS. 5a to 6c further elaborate on the model integration/fusion process(es) as described with reference to FIGS. 1a to 4i based on the specific types of multi-agent models AM 420-1 to 420-z as described with reference to FIGS. 4a to 4i.

FIG. 5A is a schematic diagram illustrating an example model fusion system 500 according to some embodiments of the invention. For simplicity, the reference numerals of FIGS. 4a to 4i may be reused for similar or the same components and the like. Furthermore, the model integration/fusion process(es) 110 and/or systems 100 as described with reference to FIGS. 1a to 3b may be further modified by the model fusion system 500 and/or model fusion process(es) as described with reference to FIGS. 5a to 6c, modifications thereof, combinations thereof and/or as herein described. Referring to FIG. 5A, let a data set D₁ be used for training an agent model (AM) M₁ 420-1 creating an agent rule base R_{M1,D1} over an agent state space S_{M1,D1} for use in modelling at least a part or portion of a real world complex system 504, or for modelling the whole of the real world complex system 504 (e.g. detection of disease and/or state of a subject from images, or other medical data of the subject). Furthermore, let a related but different data set D₂ be used for training another AM M₂ 420-z creating another agent rule base R_{M2,D2} over an agent state space S_{M2,D2} for use in modelling at least a part or portion of a real world complex system 504, or for modelling the whole of the real world complex system 504. Model integration or fusion 506 is the process or component by which D₂, a related data set in the same complex system 504 that is being modelled by M₂ 420-z, can be merged with M₁ 420-1 with the help of a semantic network S. The semantic network S may be used to determine which data sets are related to each other to increase the likelihood that the resulting AMs 420-1 and 420-z may be merged using model fusion 506. The resulting AM model, M₃=M_{1U2} 508, where U indicates union of the two AMs M₁ 420-1 and M₂ 420-z, is able to: a) Receive D₁-like input and create predictions/outputs like D₁ even in the absence M₂ inputs; b) Receive D₂-like inputs and create predictions like D₂ even in the absence M₁ inputs; c) Given an outcome from any input, predict the values missing inputs; and d) Predict outcomes when both D₁ and D₂-like inputs are present, even if the outcomes are conflicting.

For example, a real-world complex system 504 may be based on, without limitation, for example the detection and/or behaviour of a complex disease, where data associated with subjects with and/or without the complex disease may be captured in data silos. Such data may include, without limitation, for example imaging of a tumour of a plurality of subject, and blood factors of a plurality of subjects that indicate/or do not indicate the severity of the disease, and/or any other medical data and/or lifestyle data associated with subjects with and/or without the disease and the like. An AM model may be trained and built based on each data silo resulting in two or more AMs 420-1 to 420-z. For example, M1 and M2 420-1 and 420-z may therefore be independent AM models that have predictive power, however they represent two different (perhaps related) aspects of the real-world complex system 504. The process of model fusion 506 is a computational solution that respects the behaviour of each AM model constituent M1 and M2 to create a single more representative model M3 that is a merging of both models M1 and M2 at a structural and fundamental level rather than a simple merging of model outputs.

As described with reference to FIG. 5A, the model fusion process 506 is based on determining whether two or more data sets of multiple data silos that are used to build the corresponding AMs 420-1 to 420-z are related with each other in some manner. This assists in determining which data silos may be used to generate each of the AMs 420-1 to 420-z as described with reference to FIGS. 1A to 4I resulting in a high likelihood that the resulting AMs 420-1 to 420-z may be fused/merged and/or integrated together to form a single more representative model or fusion model that more accurately models and/or predicts the outputs/observables associated with the complex system 506 being modelled by the AMs 420-1 to 420-z.

FIG. 5B is a schematic diagram illustrating an example semantic network/model 510 for use with the model fusion 506 and model fusion system 500 of FIG. 5A according to the invention. In this example, the semantic network/model 510 describes a list of entities or objects 511 such as tumour 511a, cells 511b, and tissue 511c, in which the tumour 511a is part of tissue 511c and is composed of cells 511b. The semantic model 510 further includes a list of behaviours 512 and also connections between the entities/objects 511 and the behaviours. For example, the behaviour Cells divide or cell division 512b, which is a behaviour that is also manifested as the behaviour tumour growth 512a. Thus, a semantic network 510 may be built between the tissue, tumour, and cell objects 511a-511c using the corresponding behaviour objects 512 and making connections therebetween such as connections associated with non-physical behaviour, part of physical behaviour, or exhibits non-physical behaviour. Thus, if there is sufficient depth in each of the training data sets and each of the training data sets may be associated with an object/behaviour of a semantic model 510, then each training AM model 420-1 to 420-z, when optimised, will develop rules and states where each of these concepts are represented within the semantic network 510.

In essence, a semantic network 510 is the bridge between the abstract metamodel (i.e. a trained AM, also known as a kernel) and the real world. The basic foundation of the semantic model is: i) A list of entities (real world or concepts); ii) A list of connections between those entities that define, without limitation, for example, non-physical connectivity, (e.g. the computation of one concept includes/consumes the computation of another concept), and/or physical connectivity (e.g. an entity is part of or entirely consumed physically by another entity). It is noted that the term computation is used relatively loosely, for example, length/width of sepals and petals of a flower do not “compute” its species, but there is a statistical significance between these features and species, which can be approximated by a computation. Entities and their corresponding inter-connections of a semantic model 510 are related to the AS rules of one or more trained AM(s) 420-1 to 420-z when trained on a data set associated with an object/behaviour and/or connection associated with the semantic model such that there exists a set of rules, R_{E}, for entity set E, of the semantic model which each of the AM(s) may also metamodel. Thus, a semantic model may be used to identify the corresponding data sets of one or more data silos that may be useful to train and generate multiple trained AM models or kernels 420-1 to 420-z each of which model one or more aspects of a complex system 504, but which are related to each other as outlined by the semantic model, which enhances the ability for the fusion process 506 to merge/integrate and/or fuse the multiple trained AM models or kernels 420-1 to 420-z into a single more representative fusion AM model that more accurately models the complex system 504.

It is noted that each trained AM 420-1 to 420-z develops a set of agent unit rules in each of the AS nodes defining the AS network of the respective AMs 420-1 and 420-z. For example, assume AM M₁ 420-1 has agent unit ruleset R_{E1} and AM M₂ 420-z has agent unit ruleset R_{E2}. Then in order to integrate or fuse these AMs 420-1 to 420-z, there are three possibilities for fusion/integration based on whether the rulesets intersect or not: 1) No Intersection: R_{E1} and R_{E2} have absolutely no intersection according to their semantic models, then there are two choices for integration: a) each of the AM models 420-1 and 420-z can be run independently with no connectivity; or b) each of the AM models 420-1 and 420-z may co-exist in the same resulting fusion model with independent state spaces but with a shared AS topology; 2) Partial Intersection: R_{E1} and R_{E2} have some intersection according to their semantic models. These should co-exist in the same fusion model and therefore the requirements for the fusion process 506 are to: a) Find the intersecting rules; b) Optimise a conflict resolution between the rules; and/or c) merge the AS nodes associated with the intersecting rules and conflict resolution; and 3) Full Intersection: R_{E1} and R_{E2} have complete intersection according to their semantic models, i.e. they are competing models. These should co-exist in the same fusion model and therefore the requirements are to: a) Optimise a conflict resolution between the rules.

Once it is determined that two or more AM models 420-1 to 420-z may possibly have partial and/or full intersections between their rulesets and those of others of the AM models 420-1 to 420-z, the fusion process 506 may then begin the merging/fusion function based on the AS network structure of each of the AM models 420-1 to 420-z. The AS network structure is essentially a graph structure with AS nodes and connections therebetween as described with reference to FIGS. 1a to 4i. Thus, a node graph matching function may be used to determine a similarity measure between at least two AM models 420-1 and 420-z based on their corresponding AS network structures. The node graph matching function, also referred to herein as XNX graph matching algorithm/function, may represent any function that takes two graphs or subgraphs as arguments and develops a similarity measure between 0 and 1, where 0 denotes complete non-similarity and 1 denotes complete similarity. The radius should also be taken into consideration, i.e. the maximum radius to search for subgraph similarity. The XNX graph matching function is used to decide how to merge two graphs together, hence can be used to decide how to merge two AS networks together. Given that each trained AM 420-1 to 420-z has a particular AS network, the XNX graph matching function may be used to decide how to merge two AM models together, e.g. AM 420-1 and AM 420-z. The XNX graph matching function may use, without limitation, for example subgraph similarity metrics, and/or a comparison matrix as the number of AS nodes in an AM 420-1 or 420-z may be usually quite small.

FIG. 5C is a schematic diagram illustrating an example merging process 520 based on using the XNX graph merge function to merging a semantic model 510 with another semantic model 522 to form a merged semantic model 524. In FIG. 5C, the graph network A of semantic model 510 is compared with the graph network B of semantic model 522. Using a similarity metric, the XNX graph merge function may determine that the graph networks A and B are similar, as the semantic objects Tissue, Tumour, Cell and Cell Division and corresponding connections enclosed in box 526 are the same, whereas the objects growth and vascularisation nodes in the graphs A and B are not. Thus, networks A and B are similar in that most nodes and arc/connections match. Thus, the objects Tissue, Tumour, Cell and Cell Division are simply copied into a merged network C, with growth and vascularisation included with added connections being deduced accordingly. As with Euclidean distance, any number of methods can be used to derive a 0 . . . 1 range metric for similarity.

In addition to the XNX graph merge function, the fusion process 506 may make use of, without limitation, for example the belief function, and/or any other suitable function. In this case, the belief function is defined as any function that takes two n-length vectors and assigns a “belief” to each. Based on the belief, it outputs a new n-length vector which could be identical to one of the inputs or a mixture of the two (e.g. average per-locus). Furthermore, along with the belief function, an AS node merge function is used for merging the AS states in each node and also the up/down connections of their respective graphs. The AS node merge function is defined to be any function that defines how two nodes from different graphs can be merged with respect to: a) The corresponding AS states in each AS node; and b) The upwards and downwards connections in their respective AS graphs/networks.

FIG. 5D is a schematic diagram illustrating an example AS network graph merge 530 for merging a first AS graph 532 and second AS graph 534 for use in the fusion process 506 according to the invention. In this example, the AS network graph merge 530 is configured to match on the middle nodes 532a and 534a of AS graphs 532 and 534, respectively. Based on the AS node merge function, two possible outcomes 536a and 536b are illustrated. These are generated based on looking at the upwards and downwards connections of the respective AS graphs and merging the AS nodes that are similar or have AU networks that are similar. That is the XNX graph merge function may be used to determine which AS nodes have similar AU networks, and then merging those AS nodes when the AU networks have greatest similarity. Bias towards a particular way of merging is a configuration for the merge function set by the user. In addition to merging the AS nodes, the AS node merge function also defines how the corresponding AS states should be merged, where in this case for the AS nodes 532a and 532b, the AS states 532b and 534b are concatenated into state 537a on the central node 536, and the associated AS rules 532c {r1, r2, r3} and AS rules 534c {r4, r5} are concatenated on the central node 536 that is being merged to associated AS rules 537b {r1, r2, r3, r4, r5}. Thus, AS nodes may be merged accordingly.

FIG. 6A is a flow diagram illustrating an example partial intersection fusion process 600 for use in the fusion process/component 506 of FIG. 5A and/or the integration/fusion process in systems 100 and process 110 according to the invention. The steps and/or functionality of the partial intersection fusion process 600 can be applied to modify the steps and/or functionality of the integration/fusion process of system 100 and/or process 110 as described with reference to FIGS. 1a to 3b and/or the fusion process 506 as described with reference to FIGS. 4a to 5d and the like. It is assumed that at least two AMs 420-1 and 420-z are mature kernels (i.e. trained AMs), denoted M₁ and M₂, and have a high degree of individual accuracy and also the rulesets are semantically determined to be able to be deeply integrated. The partial intersection fusion process 600 may include the following steps of.

In step 601, determining an intersection of rulesets based on applying the XNX graph matching algorithm to search for areas of AS network similarity between the AS networks of M₁ and M₂; In step 602, for each area of similarity above a predefined threshold, proceed to step 603, otherwise proceed to step 602a. In step 602a, determine whether search completed, if the search has completed proceed to step 606, otherwise proceed to step 601 to find other areas of AS network similarity between the AS networks of M₁ and M₂. In step 603, for each area of similarity determine whether there is an exact match between the areas of similarity, if there is an exact match then proceed to step 604, otherwise proceed to step 605. In step 604, superimpose the AS state and rules, i.e. states and rules may be concatenated and apply a belief function to the merged AS state/rules and store the merged AS state/rules for merging into a merged AS graph network. Proceed to step 602a. In step 605, if there is a partial match above the threshold, then performing the steps of, replace the area of similarity using a node graph merge function as described with respect to FIGS. 5a to 5d and apply a belief function to the merged AS state/rules and store the merged AS state/rules for merging into a merged AS graph network. Proceed to step 602a. In step 606, retrieving any stored merged AS states/rules and merge into a merged AS graph network. In step 607, retrieve initial validation data sets, and rerun the AM training process/algorithm 430 on the merged AS graph network to re-optimise the parameters. An integration optimisation requires less weight on network restructuring mutations. The integrated/fusion model must fulfil a predefined maximum error with respect to all training sets as well as validation sets. In step 608, output the updated merged AS graph network as the fusion model/integrated AM model.

FIG. 6B is a flow diagram illustrating an example full intersection fusion process 610 for use in or modifying/combining with the fusion process/component 506 of FIG. 5A, step 606-608 of process 600 of FIG. 6A and/or the integration/fusion process in systems 100 and process 110 according to the invention. The steps and/or functionality of the partial intersection fusion process 610 can be applied to modify the steps and/or functionality of the integration/fusion process of system 100 and/or process 110 as described with reference to FIGS. 1a to 3b, the fusion component/process 506 as described with reference to FIGS. 4a to 5d, and/or steps 606-608 of process 600 of FIG. 6a and the like. It is assumed that at least two AMs 420-1 and 420-z are mature kernels (i.e. trained AMs), denoted M₁ and M₂, and have a high degree of individual accuracy and also the rulesets are semantically determined to be able to be deeply integrated. In fact, it is determined intersection of rulesets based on applying the XNX graph matching algorithm to search for areas of AS network similarity between the AS networks of M₁ and M₂ means that all the areas of the AS network have a high degree of similarity, hence a full intersection exists between the AS networks of M₁ and M₂ or there is an exact match in all areas of both the AS networks. The full intersection fusion process 610 may include the following steps of.

In step 611, for each corresponding AS node of each of the AS network(s), superimpose the AS state and rules, i.e. states and rules may be concatenated and apply a belief function to the merged AS state/rules and add the merged AS state/rules to a merged AS graph network. In step 612, retrieve initial validation data sets, and rerun the AM training process/algorithm 430 on the merged AS graph network to re-optimise the parameters. An integration optimisation requires less weight on network restructuring mutations. The integrated/fusion model must fulfil a predefined maximum error with respect to all training sets as well as validation sets. In step 613, output the updated merged AS graph network as the fusion model/integrated AM model.

FIG. 6C is a flow diagram illustrating an example fusion process 620 for use with fusion process(es) 600 and 610 of FIGS. 6a and 6b and/or in the fusion process/component 506 of FIG. 5A and/or the integration/fusion process in systems 100 and process 110 according to the invention. The steps and/or functionality of the partial intersection fusion process 630 can be applied to modify the steps and/or functionality of the integration/fusion process of system 100 and/or process 110 as described with reference to FIGS. 1a to 3b and/or the fusion process 506 as described with reference to FIGS. 4a to 5d and the like. Reference is made to FIGS. 4a to 5d, where applicable. It is assumed that multiple AMs 420-1 to 420-z have mature kernels (i.e. trained AMs) and that they are configured to model aspects of the same complex system, thus, the rulesets/bases of each of the AMs 420-1 to 420-z should have at least a partial and/or full intersection with one or more others of the AMs 420-1 to 420-z. The complex system 504 and/or 400 is modelled by a plurality of agent model(s) 420-1 to 420-z, each agent model 420-1 of the plurality of agent model(s) 420-1 to 420-z are configured to model a different portion of the complex system 400 or 504. The fusion process 630 may include the following steps of:

In step 631, determine, for each agent model of the plurality of agent models, an intersecting rule set between the agent rule bases of said each agent model and any of those other agent models in the plurality of agent model(s), where the agent rule base of each of the plurality of agent model(s) intersects with at least one other agent rule base of another of the plurality of agent model(s).

In step 632, for each agent model of the plurality of agent models, merging said each agent model with each of those agent models in the plurality of agent models determined to intersect with said each agent model to form an intermediate fused or integrated agent model based on combining those one or more layer(s), AS node(s), and/or AU(s) of each of the plurality of agent models that intersect. The partial intersection fusion process 600 and/or full intersection fusion process 610 may be applied depending on the level of intersection between the agent models and/or intermediate models and the like. Perform this step until only intermediate agent models and/or one merged model is left.

In step 633, for each of the intermediate fused or integrated agent models, determine an intersection with said each other intermediate fused or integrated agent model, and merge said intersecting intermediate fused/integrated agent models. The partial intersection fusion process 600 and/or full intersection fusion process 610 may be applied depending on the level of intersection between the intermediate models and the like.

In step 634, merging each of the intermediate fused or integrated agent models to form an fusion agent model.

In step 635, updating the fusion agent model based on one or more validation and training labelled datasets associated with each of the plurality of agent models until the integrated model is validly trained.

In steps 632 and/or 634, once an agent model is merged with another agent model to form an intermediate agent model, and/or once an intermediate agent model is merged with an agent model, and/or when intermediate agent model is merged with another intermediate agent model, then the resulting merged model may be validated using datasets associated with the merged agent models that make up the resulting merged model. That is, the steps of step 635 may be performed for each of the merging(s) in steps 632 and 634 to validate the merged models prior to further merging operations and the like.

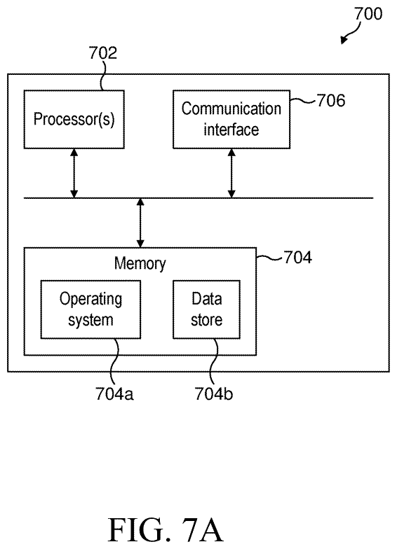

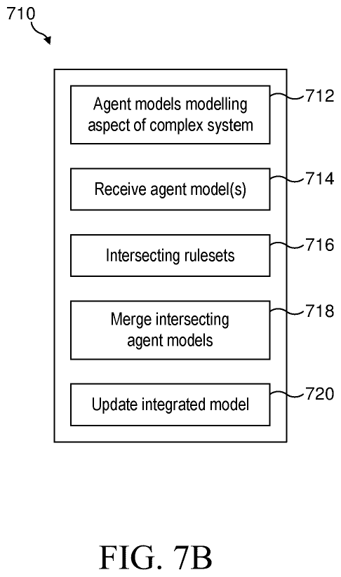

FIG. 7A is a schematic diagram illustrating an example computer apparatus/device, according to some embodiments of the invention. Referring to FIG. 7A, the computer apparatus/device 700 comprises a processor unit 702, a memory unit 704, a communications interface 706. The memory unit 704 comprises an operating system 704a and a computer-readable data storage/storage media 704b.

FIG. 7B is a schematic diagram illustrating an example model integration system, according to some embodiments of the invention. Referring to FIG. 7B, the model integration system 710 comprises an agent modelling module 712, a receiver module 714, a rule intersecting module 716, a merging module 718, and an integrated agent update module 720. The agent module 712 comprises the plurality of AMs 102a-102n, modelling aspects of the complex system 100. The receiver module 714 is configured to receive data representative of the at least two agent model(s). The rule intersection module 716 is configured to determine 112 an intersecting rule set 107a between the agent rule bases of at least a first 102a and second 102b trained agent model. The merging module 718 is configured to merge/integrate 114 the at least first 102a and second 102b trained agent model, to form an integrated agent model 109. The integrated/fusion agent model 109 is based on combining one or more layer(s), AS node(s) 108a, 108b, and/or AU(s) of the first 102a and second 102b trained agent models that correspond to the intersecting rule set 107a. The integrated agent update module 720 is configured to update 116 the integrated agent model 109 based on one or more validation and/or labelled training datasets associated with each of the at least first 102a and second 102b trained agent model(s) until the integrated model 109 is validly trained.

In another embodiment, the server may comprise a single server or network of servers. In some examples, the functionality of the server may be provided by a network of servers distributed across a geographical area, such as a worldwide distributed network of servers, and a user may be connected to an appropriate one of the network of servers based upon a user location.

In the embodiments, examples, of the invention as described above such as the above-mentioned process(es), method(s), system(s) and/or apparatus may be implemented on and/or comprise one or more cloud platforms, one or more server(s) or computing system(s) or device(s). In some instances, the above description may discuss embodiments of the invention with reference to a single user or complex system for clarity. It will be understood that in practice the system may be shared by a plurality of users, and possibly by a very large number of users simultaneously.

The embodiments described above may be fully automatic. In some examples, a user or operator of the system may manually instruct some steps of the method(s) to be carried out.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

Unless otherwise specified, all terms used herein, which include technical or scientific terms, have the same meanings that are generally understood by a person skilled in the art. It will be further understood that terms, which are defined in a dictionary and commonly used, should also be interpreted as customary in the relevant related art and not in an idealized or overly formal detect unless expressly so defined herein in various embodiments of the present invention. In some cases, even if terms are defined in the specification, they may not be interpreted to exclude embodiments of the present invention.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, subroutines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

What is claimed is:

1. A computer implemented method of detecting a disease or state of a subject from one or more images of the subject, the method comprising:
obtaining a fusion agent model configured for modelling the detection of the disease or state of the subject from one or more images of the subject, the fusion agent model derived from at least two agent model(s), each agent model trained to model the detection of the disease or state of a subject from a different imaging source, each agent model comprising:
a plurality of agent system, AS, node(s), wherein each of the AS node(s) comprise: a plurality of agent units, AUs, and a set of AS rules governing the plurality of AUs, each AU of the plurality of AUs connected to at least one other AU of the plurality of AUs;
an input layer comprising a set of AS nodes of the plurality of AS node(s);
an output layer comprising at least one AS node of the plurality of AS node(s); and
one or more intermediate layer(s), each of the intermediate layer(s) comprising another set of AS node(s) of the plurality of AS node(s);
each agent model is trained to model the detection of the disease or state of a subject based on a corresponding labelled training dataset comprising images from, and said each agent model being adapted, during training, to form an agent rule base comprising one or more sets of agent system rules; and
an agent network state comprising data representative of the interconnections between the AS nodes of the input, output and intermediate layer(s), wherein the agent rule base and agent network state are generated during training and configured for modelling said portion(s) of a complex system;
wherein obtaining the fusion agent model further comprising:
determining an intersecting rule set between the agent rule bases of the at least two agent models;
merging said at least two agent models to form a fused agent model based on combining those one or more layer(s), AS node(s), and/or AU(s) of the at least two agent models that correspond to the intersecting rule set; and
updating the fused agent model based on one or more validation and training labelled datasets associated with each of the at least two agent model(s) until the fused agent model is validly trained, wherein the trained fused agent model is the fusion agent model;
inputting said one or more images of the subject to the fusion agent model for detecting the disease or state of the subject based on the input one or more images of the subject; and
outputting data representative of an indication of whether the disease or state is detected from the one or more images of the subject.

2. The computer implemented method according to claim 1, wherein the complex system to be modelled is detection of prostate cancer of a subject that is modelled by a plurality of prostate cancer detection agent models, wherein each prostate cancer detection agent model is trained using a labelled training dataset comprising a plurality of labelled training data images of subjects in relation to detecting or recognising prostate cancer tumours from said labelled training data images, wherein each prostate cancer detection agent model uses a labelled training dataset based on images output from the same type of imaging system that is different to the imaging systems used in each of the other prostate cancer detection agent model of the plurality of prostate cancer agent models;
optionally wherein each imaging system is a particular magnetic resonance imaging, MRI, system by a particular manufacturer.

3. The computer implemented method according to claim 1, wherein the complex system is bone fracture detection of a subject that is modelled by a plurality of bone fracture detection agent models, wherein each bone fracture detection agent model is trained using a labelled training dataset comprising a plurality of labelled training data images of subjects in relation to detecting or recognising bone fractures from said labelled training data images, wherein each bone fracture detection agent model uses a labelled training dataset based on images output from the same type of imaging system, wherein each labelled training data item is annotated or labelled as to whether or not said subject of the plurality of subjects has a bone fracture, wherein each bone fracture detection agent model is trained in relation to images associated with different imaging systems.

4. The computer implemented method according to claim 1, wherein images are acquired via imaging systems or techniques based on at least one from the group of: magnetic resonance imaging, MRI; computer tomography, CT; ultrasound; or X-ray; or images from any other medical imaging system for use in detecting disease and/or state of a subject.

5. A computer implemented method of detecting a disease or state of a subject from a plurality of data sources associated with the subject, the method comprising:
obtaining a fusion agent model configured for modelling the detection of the disease or state of the subject from said plurality of data sources associated with the subject, the fusion agent model derived from at least two agent model(s), each agent model trained to model the detection of the disease or state of a subject from a different data source associated with the subject, each agent model comprising:
a plurality of agent system, AS, node(s), wherein each of the AS node(s) comprise:
a plurality of agent units, Aus, and a set of AS rules governing the plurality of Aus, each AU of the plurality of Aus connected to at least one other AU of the plurality of Aus;
an input layer comprising a set of AS nodes of the plurality of AS node(s);
an output layer comprising at least one AS node of the plurality of AS node(s); and
one or more intermediate layer(s), each of the intermediate layer(s) comprising another set of AS node(s) of the plurality of AS node(s);
each agent model is trained to model the detection of the disease or state of a subject based on a corresponding labelled training dataset derived from the corresponding data source associated with the subject, and said each agent model being adapted, during training, to form an agent rule base comprising one or more sets of agent system rules; and
an agent network state comprising data representative of the interconnections between the AS nodes of the input, output and intermediate layer(s), wherein the agent rule base and agent network state are generated during training and configured for modelling said portion(s) or aspects of a complex system;
wherein obtaining the fusion agent model further comprising:
determining an intersecting rule set between the agent rule bases of the at least two agent models;
merging said at least two agent models to form a fused agent model based on combining those one or more layer(s), AS node(s), and/or AU(s) of the at least two agent models that correspond to the intersecting rule set; and
updating the fused agent model based on one or more validation and training labelled datasets associated with each of the at least two agent model(s) until the fused agent model is validly trained, wherein the trained fused agent model is the fusion agent model;
inputting data representative of said one or more data sources associated with the subject to the fusion agent model for detecting the disease or state of the subject; and
outputting data representative of an indication of whether the disease or state is detected from the input one or more data sources associated with the subject.

6. The computer implemented method according to claim 5, wherein the complex system to be modelled is liver disease detection of a subject that is modelled by a plurality of liver disease detection agent models, wherein each liver disease detection agent model is trained using a labelled training dataset derived from a different data source associated with a plurality of subjects, said each labelled training dataset comprising a plurality of labelled training data items based on the different data source and annotated in relation to whether or not said plurality of subjects have liver disease, said each trained liver disease detection agent model associated with a different, but related, aspect of the complex system of liver disease detection.

7. The computer implemented method according to claim 6, wherein the plurality of liver disease detection agent models comprises at least the liver disease detection agent models from the group of:
a first liver disease detection agent model trained based on a labelled training dataset comprising a plurality of labelled training data items associated with a plurality of subjects, each training data item corresponding to data representative of lifestyle and/or ethnic background data of a subject of the plurality of subjects and annotated or labelled as to whether or not said subject of the plurality of subjects has liver disease;
a second liver disease detection agent model trained based on a labelled training dataset comprising a plurality of labelled training data items associated with a plurality of subjects, each training data item corresponding to data representative of the genetics of a subject of the plurality of subjects and annotated or labelled as to whether or not said subject of the plurality of subjects has liver disease;
a third liver disease detection agent model trained based on a labelled training dataset comprising a plurality of labelled training data items associated with a plurality of subjects, each training data item corresponding to data representative of one or more proteomic blood markers of a subject of the plurality of subjects and annotated or labelled as to whether or not said subject of the plurality of subjects has liver disease;
a fourth liver disease detection agent model trained based on a labelled training dataset comprising a plurality of labelled training data items associated with a plurality of subjects, each training data item corresponding to data representative of medical history of a subject of the plurality of subjects and annotated or labelled as to whether or not said subject of the plurality of subjects has liver disease;
a fifth liver disease detection agent model trained based on a labelled training dataset comprising a plurality of labelled training data items associated with a plurality of subjects, each training data item corresponding to data representative of a sonograph and/or imaging of the liver of a subject of the plurality of subjects and annotated or labelled as to whether or not said subject of the plurality of subjects has liver disease; and
one or more other liver disease detection agent model(s), each trained based on a labelled training dataset comprising a plurality of labelled training data items associated with a plurality of subjects, each training data item corresponding to data representative of modelling another aspect of the complex system for diagnosing liver disease and annotated or labelled as to whether or not said subject of the plurality of subjects has liver disease.

8. A computer implemented method of fusing or integrating at least two agent model(s) for modelling a complex system, each agent model comprising:
A plurality of agent system, AS, node(s), wherein each of the AS node(s) comprise a plurality of agent units, AUs, and a set of AS rules governing the plurality of AUs, each AU of the plurality of AUs connected to at least one other AU of the plurality of AUs;
an input layer comprising a set of AS nodes of the plurality of AS node(s);
an output layer comprising at least one AS node of the plurality of AS node(s); and
one or more intermediate layer(s), each of the intermediate layer(s) comprising another set of AS node(s) of the plurality of AS node(s);
wherein each agent model is trained to model one or more portion(s) of the complex system using a corresponding labelled training dataset, said each agent model being adapted, during training, to form:
an agent rule base comprising one or more sets of AS rules; and
an agent network state comprising data representative of the interconnections between the AS nodes of the input, output and intermediate layer(s), wherein the agent rule base and agent network state are generated during training and configured for modelling said portion(s) of the complex system;
the method comprising:
determining an intersecting rule set between the agent rule bases of at least a first trained agent model and a second trained agent model;
merging said at least first and second trained agent models to form an integrated agent model based on combining those one or more layer(s), AS node(s), and/or AU(s) of the first and second trained agent models that correspond to the intersecting rule set; and
updating the integrated agent model based on one or more validation and training labelled datasets associated with each of the at least first and second trained agent model(s) until the integrated model is validly trained.

9. The computer implemented method according to claim 1, wherein the complex system is modelled by a plurality of agent model(s), each agent model of the plurality of agent model(s) configured to model a different portion of the complex system, the method further comprising:
determining an intersecting rule set between two or more of the agent rule bases of the plurality of agent model(s), wherein the agent rule base of each of the plurality of agent model(s) intersects with at least one other agent rule base of another of the plurality of agent model(s); and
merging said plurality of agent models to form an integrated agent model based on combining those one or more layer(s), AS node(s), and/or AU(s) of each of the plurality of agent models that correspond to the intersecting rule set; and
updating the integrated agent model based on one or more validation and training labelled datasets associated with each of the at least first and second trained agent model(s) until the integrated model is validly trained.

10. The computer implemented method according to claim 1, wherein the complex system is modelled by a plurality of agent model(s), each agent model of the plurality of agent model(s) configured to model a different portion of the complex system, the method further comprising:
for each agent model of the plurality of agent models, determining an intersecting rule set between the agent rule bases of said each agent model and any of those other agent models in the plurality of agent model(s), wherein the agent rule base of each of the plurality of agent model(s) intersects with at least one other agent rule base of another of the plurality of agent model(s); and
for each agent model of the plurality of agent models, merging said each agent model with each of those agent models in the plurality of agent models determined to intersect with said each agent model to form an intermediate fused or integrated agent model based on combining those one or more layer(s), AS node(s), and/or AU(s) of each of the plurality of agent models that intersect;
merging each of the intermediate fused or integrated agent models to form an fusion agent model; and
updating the fusion agent model based on one or more validation and training labelled datasets associated with each of the plurality of agent models until the integrated model is validly trained.

11. The computer implemented method according to claim 1, wherein determining an intersecting rule set between at least the first trained agent model and second trained agent model further includes determining a compatibility score between at least the first trained agent model and the second trained agent model, and indicating those models of at least the first and second trained agent models to be merged when the compatibility score is above a predetermined threshold;
optionally wherein calculating the compatibility score comprises determining whether one or more semantic relationships exist between at least the first trained model and at least the second trained model;
optionally wherein determining whether one or more sematic relationships exist further comprises forming a semantic network between at least the first trained model and the second trained model, wherein interconnections in the semantic network exist when one or more entities associated with the first trained model are connected, correlate or have a relationship with one or more entities associated with the second trained model.

12. The computer implemented method according to claim 1, the steps of determining an intersection rule set and merging at least the first trained agent model and at least the second trained agent model further comprising:
determining one or more areas of similarity between agent state networks of at least the first trained agent model and second trained agent model;
comparing, based on each area of similarity, the AS rule sets of the AS nodes in the area of similarity between at least the first trained agent model and the second trained agent model; and
merging, based on the comparison of each area of similarity, the corresponding AS nodes and interconnections between the layers of at least the first and second trained models;
optionally wherein the method further comprises:
determining, using a graph matching algorithm, the one or more areas of similarity between at least the first trained agent model and the second trained agent model; and
merging the corresponding AS nodes and interconnections further comprising:
concatenating, based on the determined areas of similarity, the corresponding sets of AS rules and AS node states of the at least first trained agent model and the second trained agent model; and
applying a belief function to the concatenated set of AS rules and AS states.

13. The computer implemented method according to claim 1, further comprising:
training each agent model to model one or more portions of the complex system using a labelled training dataset comprising a plurality of labelled training data items corresponding to the one or more portions of the complex system, wherein interconnections between AS nodes are initially randomised, training each agent model further comprising:
receiving each labelled training data item from a source and vectorising each received labelled training data item;
processing, by at least one of the input, intermediate and output layer(s), each vectorised training data item by the corresponding AS node(s), wherein the AS node(s) are located in the same or different layers and perform at least one of a plurality of functions;
outputting, from the output layer, an output vector for each labelled training data item in the labelled training dataset based on the processed vectorised training data item; and
updating the AS node(s) of at least one of the input, intermediate layer(s) based on comparing each output vector with each corresponding labelled training data item.

14. The computer implemented method according to claim 13, wherein:
receiving and vectorising each labelled training data item further comprising receiving each labelled training data item and converting each labelled training data item into an input training data vector of a predetermined size, wherein the input training data vector includes feature elements associated with the training data item and two or more elements representing the label;
processing each vectorised training data item further comprising:
propagating one or more portions of each input training data vector to one or more AS(s) of the input layer, wherein each AS node uses a plurality of AUs to process the propagated corresponding one or more portions of each input training data vector for outputting an input AS node output vector;
propagating each input AS node output vector from each of the AS node(s) of the input layer to correspondingly connected downstream AS node(s) of at least one of the intermediate and output layer(s), wherein each downstream AS node processes one or more propagated input AS node output vector(s) using the corresponding plurality of AUs and outputs a downstream AS node output vector(s); and
iteratively propagating each downstream AS node output vector to correspondingly connected further downstream AS node(s) of at least one of the intermediate and output layer(s) for processing and outputting further downstream AS node output vector(s) until all of the AS node(s) of the output layer receive all of those downstream AS node output vector(s) from the corresponding connected AS nodes of said at least one intermediate layer;
outputting, from the output layer, an output vector for each labelled training data item further comprising:
outputting an output AS node vector corresponding to each labelled training data item based on processing, by the one or more AS node(s) of the output layer, those received downstream AS node output vector(s) associated with said each labelled training data item;
interpreting or classifying the output AS node vector to form a predicted label associated with the output AS node vector;
updating the AS node(s) of at least one of the input, intermediate and output layer(s) further comprising:
evaluating, for each output AS node vector corresponding to each labelled training data item, an indication of an error between the predicted label associated with the output AS node vector and the label associated with the corresponding labelled training data item using a cost function; and
performing a minor mutation of the agent network state and agent rule base based on the indication of the error;
repeating the receiving and vectorising, processing, outputting and updating steps for each labelled training data item of the labelled training data set until one or more of:
a minimum error rate is achieved for all the labelled training data items of the labelled training dataset;
a set number of training epoch cycles of the labelled training dataset is achieved;
a set number of training cycles for each labelled training data item is achieved; and
in response to a set number of epoch cycles being met and an error rate for all the labelled training data items being greater than the minimum error rate, then performing a major mutation of the agent network state and agent rule base based on mutating the interconnection topology of the agent network state and/or one or more AS rules of the agent rule base and repeating the training steps of: receiving and vectorising, processing, outputting and updating steps for each labelled training data item of the labelled training data set.

15. The computer implemented method according to claim 13 wherein, prior to processing one or more input vector(s), each AS node waits for all AS nodes connected to said each AS node to send the corresponding one of the one or more input vector(s), and process(es) said one or more input vector(s) once they all have been received.

16. The computer implemented method according to claim 13, further comprising, once an AS node sends the corresponding output vector towards one or more connected AS node(s), sending by said AS node the output vector to each one or more upstream AS node(s) connected to said AS node;
optionally wherein said each upstream AS node reduces a threshold for outputting an output vector.

17. The computer implemented method according to claim 13, wherein:
the plurality of agents of each AS node includes a designated input agent for receiving vectors from one or more upstream AS nodes connected to said each AS node and a designated output agent for propagating an output vector to one or more downstream AS nodes connected to said each AS node;
each agent of the plurality of agents includes a set of agent rules from an agent rule base, the set of agent rules being the same for each agent of the plurality of agents;
each of the agents of the plurality of agents operates on identically sized vectors, the vectors of each of the plurality of agents defining a vector state space or AS node state space;
iteratively processing the input agent vectors of each agent received from those other agents connected to said each agent until a maximum number of iterative cycles based on the agent rule set, an activation threshold value modified by an activation threshold function in each cycle, and a current activation value modified by a current activation value in each cycle;
outputting an agent vector for input to one or more other agents connected to said each agent when the current activation value satisfies the activation threshold function; and
modifying activation threshold function downward to current activation value when the activation value is less than the activation threshold.

18. The computer implemented method according to claim 1, wherein each agent of the plurality of agent(s) of an AS node has a local state vector;
optionally wherein a value of the local state vector is updated after an iteration of a cycle and/or is set based on a historical value;
optionally further comprising: processing, the vectorised data at the input layer comprises:
determining a firing threshold at the input AS node based on the received one-dimensional data;
computing, at the input AS node in the input layer, a transformation of the one-dimensional input vector to a first vector of first size based on the firing threshold of the input AS node; and
transmitting or propagating, from the input layer to the one or more intermediate layer, the first vector to each agent of the plurality of AS nodes.

19. The computer implemented method according to claim 13, wherein vectorising the received data further comprises splicing the received data into a one-dimensional input vector based on one or more of: propagating the one-dimensional input vector to each AS node of the input layer; dividing the one-dimensional input vector into one or more portions, wherein each portion is propagated to a different AS node of the input layer; or applying a sliding window of a fixed length over the one-dimensional vector for propagating corresponding fixed length portions of the one-dimensional vector to a different AS node of the input layer.

20. The computer implemented method according to claim 1, wherein each AS node of the one or more intermediate and output layer(s) is coupled to a select/reduce function component configured for receiving each of the one or more output vectors from one or more upstream AS node(s) connected to said each AS node, wherein the select/reduce function component combines or transforms the received one or more output vectors into an input vector for input to said each AS node;
optionally wherein collating, using the S/R function, the first vector comprises: comparing, at an AS, a length of the received first vector with a input vector local to the AS, selecting, a sub-set of values that are common to the first vector, and reducing the selected sub-set of values with a single value.
